(19) United States
(12) Patent Application Publication
(10) Pub. No.: US 2025/0380973 A1
(43) Pub. Date: Dec. 18, 2025

(54) COMPLIANT BONE PLATE FOR FRACTURE FIXATION

(71) Applicant: The Penn State Research Foundation, University Park, PA (US)

(72) Inventors: Connor Huxman, University Park, PA (US); Jared Butler, University Park, PA (US); April Armstrong, University Park, PA (US); Gary Updegrove, University Park, PA (US); Gregory Lewis, University Park, PA (US)

(21) Appl. No.: 19/130,429

(22) PCT Filed: Nov. 16, 2023
(86) PCT No.: PCT/US2023/080052

§ 371 Date: May 15, 2025

Related U.S. Application Data
(60) Provisional application No. 63/425,843, filed on Nov. 16, 2022.

(51) Int. Cl. A61B 17/80; A61B 17/68

(52) U.S. Cl. CPC A61B 17/8009; A61B 17/8052; A61B 2017/681

(57) ABSTRACT

A compliant bone plate that can be to be attached to two or bone fragments has a frame, a first suspended fixation body, a first flexure, and a second flexure. The frame forms therethrough one or more frame apertures, and the inner wall of the frame formed by a first frame aperture of the one or more frame apertures includes an upper wall, a lower wall, a right side wall, and a left side wall. The first suspended fixation bod is within a first frame aperture of the one or more of the one or more frame apertures, and the outer face of the first suspended fixation body includes a top face, a bottom face, a right face, and a left face. The first flexure connects the right side wall of the frame to the right face of the first suspended fixation body, and the second flexure connects the left side wall of the frame to the left face of the first suspended fixation body.

### CROSS-REFERENCE TO RELATED APPLICATIONS

This application claims the benefit of U.S. Provisional Patent Application Ser. No. 63/425,843 titled Compliant Bone Plate for Fracture Fixation filed Nov. 16, 2022, which is hereby incorporated herein by reference in its entirety.

### TECHNICAL FIELD

The present disclosure relates to devices for treating bone fractures and, more particularly, compliant bone plate for fracture fixation.

### BACKGROUND

In the United States, there are over a million long bone fractures each year. An estimated 12% of these fractures result in non-unions, creating an approximate added annual medical expense of $4.6 billion. In recent decades, it has been established that for optimal secondary healing of long bone fractures, a controlled amount of micromotion, or axial strain, should be present between bone fragments, allowing for dynamic stimulation of bone to improve fracture healing. Despite this knowledge, rigid metallic bone plates remain the default option to treat bone fractures. These fixation plates, though familiar to surgeons, are far stiffer than healing cortical bone, resulting in minimal and asymmetrical motion at the fracture site, which can then lead to mal-unions or non-unions, in which the bone fails to heal properly. As illustrated in FIGS. 1A and 1B, interfragmentary motion delivered by traditional rigid plates is often minimal and asymmetrical, with motion primarily resulting from plate bending.

This suppressed and asymmetrical interfragmentary motion can then lead to reduced mechanical strength of healed bones and high non-union rates-reported as high as 15% for humeral shaft fractures alone. If a fixation plate were capable of elastically expanding and compressing, it could allow for the dynamic stimulation of bone while providing sufficient support while the fracture heals.

Compliant mechanisms offer a promising alternative direction for the design of orthopedic implants. Compliant mechanisms are devices that obtain their motion through the deflection of elastic members. In contrast to existing technologies aimed to reduce axial stiffness (such as sliding or rotating parts), flexible members can reduce wear, reduce maintenance, and provide predictable behavior.

The present disclosure is directed to overcoming these and other problems of the prior art by providing a non-rigid, mechanically compliant bone plate for long bone fractures to stimulate bone fragments for improved healing. The mechanically compliant bone plate described herein can reduce the likelihood of financially costly and technically challenging revision surgery having increased operative risk. Improved healing rates would allow patients to return to normal activities sooner, and because the compliant bone plate can be designed to be manufactured from a single part, production costs can be minimized. The disclosed technology can improve fixation between the plate and screws since no sliding parts are required for translation. Additionally, with the ability to use the compliant bone plate to provide continuous compression for primary fracture healing, the surgeon would have the ability to utilize locking screw technology, which can improve fixation in poor bone quality. Furthermore, the compliant bone plate can have a low axial stiffness while also having a high bending and torsional stiffness, which provides the mechanical behavior that bone needs to heal. The compliant bone plate can address problems of using low-modulus plates, which reduce axial stiffness at the expense of reducing bending and torsional stiffness.

### SUMMARY

Embodiments of the present disclosure may address and overcome one or more of the above shortcomings and drawbacks by providing a compliant bone plate for fracture fixation.

In an exemplary embodiment, a compliant bone plate that can be to be attached to two or bone fragments includes a frame, a first suspended fixation body (“SFB”), a first flexure, and a second flexure. The frame forms therethrough one or more frame apertures, and the inner wall of the frame formed by a first frame aperture of the one or more frame apertures includes an upper wall, a lower wall, a right side wall, and a left side wall. The first suspended fixation bod is within a first frame aperture of the one or more of the one or more frame apertures, and the outer face of the first SFB includes a top face, a bottom face, a right face, and a left face. The first flexure connects the right side wall of the frame to the right face of the first SFB, and the second flexure connects the left side wall of the frame to the left face of the first SFB.

In some embodiments, a respective axial movement of the first SFB within the first frame aperture is limited by a distance between the outer face of the first SFB and the inner wall of the first frame aperture. In some embodiments, the first SFB is configured to attach to a first of the at least two bone fragments, and the frame is configured to attach to a second of the at least two bone fragments. In some embodiments, the compliant bone plate also includes a second SFB within a second of the one or more frame apertures configured to attach to a second of the at least two bone fragments. In some embodiments, the first SFB forms therethrough a plurality of attachment apertures.

In some embodiments the first SFB forms therethrough a first set of one or more attachment apertures and is configured to attach to a first of the at least two bone fragments, and the compliant bone plate further includes a second SFB within one of the one or more frame apertures. The second SFB forms therethrough a second set of one or more attachment apertures and is configured to attach to a second of the at least two bone fragments, and the compliant bone plate is configured to be attached to the at least two bone fragments only at the one or more attachments apertures in the first and second SFBs.

In some embodiments, the compliant bone plate is configured to promote secondary healing of the at least two bone fragments by permitting axial movement of the first SFB within the first frame aperture. In some embodiments, the first and second flexures can be selectively placed from a first configuration in which the first and second flexures are at rest to a second configuration in which the first and second flexures are pre-strained, and when the compliant bone plate is installed with the first and second flexures in the second configuration, the compliant bone plate promotes primary healing by compressing the at least two bone fragments together as the first and second flexures return to the first configuration.

In some embodiments, the first SFB forms therethrough a cutout, and at least one of the first and second flexures is connected to the first SFB in the cutout. In some embodiments, the compliant bone plate also includes a lateral guiding rod extending through at least part of the frame and at least part of the first SFB. In some embodiments, each of the frame and the first SFB comprise a respective thicker first area and a respective thinner second area and the compliant bone plate further includes a support plate proximate the respective thinner second areas of the frame and the first SFB to prevent out-of-plane rotation of one or more of the first SFB relative to the frame.

In another exemplary embodiment, a compliant bone plate that can be to be attached to at least two bone fragments includes a frame forming therethrough one or more frame apertures; one or more suspended fixation bodies (“SFBs”) within one or more of the one or more frame apertures, wherein each of the one or more of the SFBs form therethrough one or more attachment apertures, and wherein each of the one or more SFBs configured to attach to a respective bone fragment such that no more than one of the one or more SFBs is attached to a respective bone fragment of the at least two bone fragments; and one or more flexures, each flexure connecting a respective one of the one or more SFBs to the frame. The compliant bone plate is configured to be attached to the at least two bone fragments only at the one or more attachments apertures in the one or more SFBs.

In some embodiments, each of the one or more SFBs is within a respective frame aperture, and a respective axial movement of a respective SFB within a respective frame aperture of the one or more frame apertures is limited by a distance between a respective outer face of the respective SFB and a respective wall of the respective frame aperture. In some embodiments, at least one of the one or more SFBs forms therethrough a plurality of attachment apertures. In some embodiments, an inner wall of the frame formed by a first of the one of the one or more frame apertures and includes an upper wall, a lower wall, a right side wall, and a left side wall; the one or more SFBs includes a first SFB within a first frame aperture of the one or more frame apertures, wherein an outer face of the first SFB includes a top face, a bottom face, a right face, and a left face; and the one or more flexures includes a first flexure connecting the right side wall of the frame to the right face of the first SFB, and a second flexure connecting the left side wall of the frame to the left face of the first SFB. In some embodiments, a first SFB of the one or more SFBs forms therethrough a cutout, and at least one of the one or more flexures is connected to the first SFB in the cutout.

In yet another exemplary embodiment, a compliant bone plate that can be attached to at least two bone fragments includes a frame forming therethrough one or more frame apertures; one or more suspended fixation bodies (“SFBs”) within one or more of the one or more frame apertures; and one or more flexures, each flexure connecting a respective one of the one or more SFBs to the frame, wherein the one or more flexures comprise at least one of a switchback turn, an LET joint, a curved portion, and a plurality of linear flexures in parallel.

In some embodiments, each of the one or more SFBs is within a respective frame aperture, and a respective axial movement of a respective SFB of the one or more SFBs within a respective frame aperture of the one or more frame apertures is limited by a distance between a respective outer face of the respective SFB and a respective wall of the respective frame aperture. In some embodiments, each of the one or more SFBs configured to attach to a respective bone fragment such that no more than one of the one or more SFBs is attached to a respective bone fragment of the at least two bone fragments, and each of the one or more SFBs forms therethrough a plurality of attachment apertures and the compliant bone plate is configured to attach to the at least two bone fragments only at the plurality of attachments apertures in the one or more SFBs. In some embodiments, an inner wall of the frame formed by a first of the one of the one or more frame apertures and includes an upper wall, a lower wall, a right side wall, and a left side wall; a first SFB of the one or more SFBs within a first frame aperture of the one or more frame apertures, wherein an outer face of the first SFB includes a top face, a bottom face, a right face, and a left face; and the one or more flexures includes a first flexure connecting the right side wall of the frame to the right face of the first SFB, and a second flexure connecting the left side wall of the frame to the left face of the first SFB.

This summary is provided to introduce a selection of concepts in a simplified form that are further described below in the detailed description. This summary is not intended to identify key features or essential features of the claimed subject matter, nor is it intended to be used to limit the scope of the claimed subject matter. Additional features and advantages of the disclosed technology will be made apparent from the following detailed description of illustrative embodiments that proceeds with reference to the accompanying drawings.

### BRIEF DESCRIPTION OF THE DRAWINGS

The foregoing and other aspects of the present disclosure are best understood from the following detailed description when read in connection with the accompanying drawings. For the purpose of illustrating the disclosed technology, there are shown in the drawings embodiments that are presently preferred, it being understood, however, that the disclosed technology is not limited to the specific instrumentalities disclosed. Included in the drawings are the following Figures:

FIGS. 1A and 1B illustrate interfragmentary motion delivered by traditional rigid bone plates.

FIG. 2 illustrates an embodiment of a compliant bone plate having two suspended fixation bodies, according to an embodiment of the disclosure.

FIG. 3 illustrates an embodiment of a compliant bone plate with screw holes in the outer frame, according to an embodiment of the disclosure.

FIGS. 4A and 4B illustrate how a compliant bone plate can be used to target primary healing, according to an embodiment of the disclosure.

FIGS. 5A and 5B illustrate how a compliant bone plate can be used to target secondary healing, according to an embodiment of the disclosure.

FIGS. 6A-6D illustrate a long bone fracture fixed with a flexure-based compliant bone plate, according to an embodiment of the disclosure.

FIGS. 7A-7C illustrate how a straight flexure can undergo axial deflection, according to an embodiment of the disclosure.

FIGS. 8A-8G illustrate various embodiments of compliant bone plates with straight flexures, according to embodiments of the disclosure.

FIGS. 9A-9D are graphs that model the displacement of straight flexures, according to an embodiment of the disclosure.

FIGS. 10A-10C and 11 illustrate various embodiments of compliant bone plates having a plurality of straight flexures in parallel, according to embodiments of the disclosure.

FIGS. 12A-12F illustrate various embodiments of compliant bone plates with switchback flexures, according to embodiments of the disclosure.

FIG. 13 illustrates an embodiment of a compliant bone plate having step-like features and switchback flexures, according to an embodiment of the disclosure.

FIGS. 14A-14N illustrate how serpentine flexures can be modeled with mathematic equations to determine optimal geometric parameters, according to an embodiment of the disclosure.

FIGS. 15A-15G show example dimensions and axial loading simulation results for the present disclosure consisting of straight flexures or serpentine flexures with a target axial range of motion of 0.3 mm, according to embodiments of the present disclosure.

FIG. 16 illustrates a compliant bone plate with multiple types of compliant elements, according to an embodiment of the disclosure.

FIGS. 17A and 17B illustrate various embodiments of compliant bone plates having flexures with an increased thickness, according to embodiments of the disclosure.

FIGS. 18A and 18B illustrate various views of an embodiment of a compliant bone plate having lamina emergent torsional (LET) joint flexures, according to an embodiment of the disclosure.

FIGS. 19A-19F illustrate various embodiments of compliant bone plates having curved-member flexures to be elongated, according to embodiments of the disclosure.

FIG. 20 illustrates an embodiment of a compliant bone plate having long, segmented flexures with switchback elements in series, according to an embodiment of the disclosure.

FIG. 21 illustrates an embodiment of a compliant bone plate having lattice- or polygon-inspired patterns, according to an embodiment of the disclosure.

FIG. 22 illustrates an embodiment of a compliant bone plate having triangular-feature flexures, according to an embodiment of the disclosure.

FIGS. 23A-23C illustrate the various flexures and their transverse and torsional stiffness, according to embodiments of the present disclosure.

FIGS. 24A-24D illustrate flexures having fixed-free and pinned-pinned boundary conditions, according to embodiments of the disclosure.

FIGS. 25A-25E illustrate embodiments of compliant bone plates having pre-curved flexures, according to embodiments of the disclosure.

FIGS. 26A and 26B illustrate a method of using a compliant bone plate according to the embodiments described herein to facilitate primary healing, according to an embodiment of the disclosure.

FIG. 27 illustrates an embodiment of a compliant bone plate having ratcheting features for maintaining compression, according to an embodiment of the disclosure.

FIGS. 28A and 28B illustrate various views of an embodiment of an asymmetrical compliant bone plate, according to an embodiment of the disclosure.

FIGS. 29A-29E show how, a rigid, thin support plate can be added via pins, welding, or other manufacturing techniques or be designed directly into the static outer frame, according to an embodiment of the disclosure.

FIGS. 30A-30E show bone plates incorporating multiple rigid support plates connected to the static outer frame, according to embodiments of the present disclosure.

FIGS. 31A-31F show embodiments and results for support plate feature, according to an embodiment of the disclosure.

FIGS. 32A-32C illustrate an embodiment of a compliant bone plate having a dual cover, according to an embodiment of the disclosure.

FIG. 33 illustrates an embodiment of a compliant bone plate being inserted into a rigid outer shell, according to an embodiment of the disclosure.

FIGS. 34A-34H illustrate the need for a compliant bone plate with angled cuts, according to an embodiment of the present disclosure.

FIGS. 35A-35F illustrate various views of a compliant bone plate comprising longitudinal slots, according to an embodiment of the disclosure.

FIGS. 36A-36F illustrate various views of a compliant bone plate comprising a transverse pin, according to an embodiment of the disclosure.

FIGS. 37A-37G illustrate embodiments of a compliant bone plate without an outer frame, according to an embodiment of the disclosure.

FIGS. 38A-38E illustrate compliant plates which affix to bone with methods other than screws, according to embodiments of the disclosure.

FIGS. 39A and 39B illustrate a compliant bone plate configured to wrap around bone, according to an embodiment of the present disclosure.

FIGS. 40A-40D illustrates a compliant bone plate with (FIGS. 40A-40C) and without (FIG. 40D) an insert in the negative regions around the suspended fixation body, according to embodiments of the present disclosure.

FIGS. 41A and 41B illustrate embodiments of modular (“puzzle piece”) flexible compliant bone plates, according to embodiments of the present disclosure.

FIGS. 42A and 42B illustrate an embodiment of a compliant plate that incorporates multiple modular components, according to an embodiment of the disclosure.

FIG. 43 illustrates an embodiment of a compliant plate with multiple materials, according to an embodiment of the disclosure.

FIG. 44 shows a compliant metaphyseal bone plate, according to an embodiment of the present disclosure.

FIGS. 45A and 45B show various views of a crescent cross-section compliant plate, according to an embodiment of the present disclosure.

FIGS. 46A-46C illustrate various views of an embodiment of a compliant bone plate having limited contact features, according to embodiments of the disclosure.

FIGS. 47A-47C show screw holes consisting of either locked (threaded) holes, conventional compression (chamfered countersunk) holes, or conventional oblong compression (chamfered countersunk) holes, respectively, and according to embodiments of the present disclosure.

FIGS. 48A-48E illustrate examples of manufacturing methods for manufacturing a compliant bone plate, according to embodiments of the disclosure.

FIG. 49 shows a compliant bone plate with two suspended fixation bodies within a single hole in an outer frame, according to an embodiment of the present disclosure.

FIGS. 50A-50C show various ways one or more support plates can be connected to a compliant bone plate, according to embodiments of the present disclosure.

### DETAILED DESCRIPTION

The present disclosure describes a mechanically compliant bone plate. It can deliver controlled axial motion between fractured bone fragments which is known to stimulate secondary bone healing or compression which is known to stimulate primary healing. The disclosed technologies address limitations associated with conventional locking plate fixation as well as some alternative flexible plate fixation methods.

Fractured bones heal through one of two biological pathways: primary healing, in which compression with very little motion is achieved and maintained between bone fragments; and secondary healing, in which a fracture gap remains between bone fragments and interfragmentary motion stimulates the formation and solidification of a callus across the fracture site resulting in healing bone. The present disclosure describes embodiments of a novel fracture fixation plate that can achieve healing via either biological healing pathway.

Bone fractures treated with traditional osteosynthesis locking plates typically use a locking mechanism within the screws or outside of the screw (i.e. a locking cap over a screw). Locking screws typically have a tapered threaded screw head that engages and locks to tapered threaded receiving holes in the plate. This results in an angularly stable connection between plate and screw, allowing for rigid fixation to bone without requiring the plate to be compressed to the bone. Locking plates offer advantages compared to their predecessor non-locking plates. When bone fragments are compressed well, locking plates can perform well; however, when a fracture gap remains, such as in the case of comminuted or complex fractures, locking plates have been shown to be too stiff to reliably induce the secondary healing. The evolution from non-locking to locking plates has been positive clinically, resulting in superior stability between the plate and screws, as well as increased blood flow to the fracture site since the plates are not compressed to the bone surface.

One major clinical challenge associated with traditional locking plates is the high stiffness they possess. These traditional locking plates are made from rigid pieces of stainless steel or titanium alloy which have a stiffness about an order of magnitude greater than cortical bone.

Some efforts have been made to reduce the rigidity of traditional locking plate constructs while still maintaining the advantages of increased fixation and angular stability that locking plates provide. For example, one conventional solution is a flexible plate fixation of bone fractures which involves elastically suspended screw holes. These elastic suspension methods involve either silicon components which compress under loads or machined flexible elements which elastically deflect. However, another conventional solution uses elastic suspension around individual screw holes. This results in two challenges: First, the axial stiffness of the plate becomes dependent on the number of screws used, since elastic suspension elements are only engaged and contribute to plate stiffness if they surround a screw hole that is being occupied; Second, the use of silicon elastomer members introduces multiple materials and multiple components to the plate's design which must be assembled together. These challenges around part count and material increase the design's complexity, potential production cost, and potential performance in vivo.

The present disclosure addresses these challenges of both traditional locking plates and alternative flexible bone plates. The present disclosure describes a novel concept including axially flexible bone plates consisting of an outer frame, one or more suspended bodies containing multiple screw holes, and flexible elements connecting the outer frame to the suspended bodies. By including multiple screw holes in each suspended body, the axial stiffness of the plate is independent of the number of screw holes utilized since all flexible elements are engaged. By achieving axial motion through the elastic deflection of flexible elements in the plate, as opposed to additional components consisting of a different material than the plate, the present disclosure can facilitate micromotion between bone fragments while using a material such as stainless steel-a surgeon-preferred material that may otherwise be too stiff in its traditional rigid design. Additionally, no assembly is required for many of the present disclosure's embodiments in which the plate consists of as little as one piece. By including a specified distance between the outer frame and suspended bodies, the amount of axial motion between proximal and distal screw holes in the plate, and thus the amount of axial motion between proximal and distal bone fragments, can be controlled to prevent over strain of the healing bone. As will be shown herein, the present disclosure introduces many new flexible element topologies and features for preventing excess out-of-plane rotation of the screw holes in the plate.

The present disclosure also describes embodiments for stimulating primary healing of bone fractures through interfragmentary compression. Traditional compression plating of bone fractures for primary healing involves eccentric placement of non-locking screws in countersunk screw holes, creating interfragmentary compression. With the present disclosure, the principle of pre-strain is leveraged to elastically pull apart proximal and distal screw holes, fix the plate to the bone, then release the pre-strain mechanism resulting in compression between bone fragments. A major advantage of the present disclosure for primary healing includes the ability to create interfragmentary compression while using locking screws instead of conventional non-locking screws, since locking screws can increase construct stability. Another advantage of the present disclosure for primary healing is the forgiveness for the lack of full compression immediately after plate insertion, which can be challenging clinically. The elastic pre-strain of the flexible members drives the compression of the plate and bone which can aid in the closure of fracture gaps. A further clinical advantage is the possible removal of a challenging and demanding intraoperative surgical insertion step of pre-bending plates. Pre-bending plates is often done to achieve compression at the far cortex, balancing the tendencies of these plates to achieve greater near cortex compression and less at the far cortex. The present technology may be able to off-set the deficiencies of traditional compression plating by delivering more symmetrical interfragmentary compression without the need to pre-bend plates.

The present disclosure describes a compliant bone plate for fracture fixation. As will be described in greater detail below, disclosed embodiments teach a bone plate comprising compliant mechanisms, referred to as “flexures” herein. A flexure is a connection between members that can elastically change shape.

The compliant bone plate described herein can be a plate for any bone fracture. For example, it could be used on long bone fractures, but also on odd-shaped bone like scapulas or jaws etc. Long bones include, for example, the femur, tibia, humerus, and any other long, cylindrical bone in the body. Specifically, the compliant bone plate can be used for bones that would fracture in a comminuted way, meaning three or more bone fragments, as well as a transverse fracture, an oblique fracture, a spiral fracture, a wedge fracture, or any fracture where there are two or more completely separated bone fragments.

In some embodiments, a compliant bone plate is formed by removing material from selected regions, through top-down manufacturing, thereby creating a series of compliant flexures (thin, long connections) between the distal half of the compliant bone plate and the proximal half of the compliant bone plate. This can allow the two halves to translate axially relative to one another. In another embodiment, a compliant bone plate can comprise micro- or meso-structures (i.e. small honeycomb pattern) or functional grading of different materials. (i.e. nitinol for flexible regions, and cobalt-chrome or stainless steel for rigid regions) through, in some embodiments, additive manufacturing, which would allow the plate to compress and expand axially. In yet other embodiments, a compliant bone plate comprises two portions that move relative to one another through flexures and can be guided through a contact surface or channel and a mating protruded piece so as to constrain the motion along the surfaces or channels with sliding or rolling contact. In other embodiments, a compliant bone plate comprises two portions moving relative to one another (through mechanisms such as any of the previous three embodiments, for example) with negative space regions filled with a bioresorbable foam or polymer, which provide stiffness to the compliant bone plate initially, and can degrade with time in the body creating a less stiff compliant bone plate in the weeks after insertion into the body. Using flexures in a bone plate, as described herein, provides several advantages: the compliant bone plate can be used for primary healing or secondary healing, it can be highly customizable, it can be made from a single piece of material, and it can be made of steel.

Due to its configuration, the compliant bone plate described herein could allow surgeons to change the degree or mode of healing intraoperatively without reaching for a different type of plate. The compliant bone plate can be used for primary or secondary healing and can allow for direct manipulation of the amount of compression or tension on the bone fragments without technically challenging eccentric compression screw placement or pre-bending the bone plate. Because the compliant bone plate can be used for primary or secondary healing, the surgeon does not have to make the determination of what type of healing is most appropriate (and thus what type of compliant bone plate is needed) before he or she gets into the operating room. Rather, he or she can bring one compliant bone plate into the operating room and decide then whether primary healing or secondary healing is more appropriate.

As explained above, the compliant bone plates described herein can be used for primary or secondary healing. As known in the art, primary healing occurs when two bone fragments are compressed against each other. To use the compliant bone plate for primary healing, as will be further explained below, the suspended fixation bodies can be pulled away from each other and each attached to a bone fragment. As the suspended fixation bodies attempt to return to their at rest position, the bone fragments will be brought together in compression.

Secondary healing occurs when a small amount of motion occurs between two bone fragments. To use the compliant bone plate for secondary healing, as will be further explained below, the suspended fixation bodies can each be attached to a bone fragment. Each bone fragment can move axially as the suspended fixation body moves within a hole of a frame.

In addition, as is illustrated in this disclosure, the compliant bone plates described herein can be highly customizable. When designing a compliant bone plate for a particular patient, the following variables can be selected: (i) the size of the fracture gap, (ii) the amount of interfragmentary strain, (iii) the presence and magnitude of shear motion, and (iv) the symmetry of the motion across the fracture gap. In some embodiments, axial strain should be between 10-30%, not exceeding 40%, to promote optimal callus formation, fracture gaps should remain relatively small, around 1-3 mm, shear strain should be minimized, and axial strain should be delivered symmetrically to ensure even callus formation. The following variables can be adjusted to achieve a specific biomechanical behavior (e.g., stiffness, amount of interfragmentary motion and strain): the length, width, and thickness of the frame, the suspended bodies, and the flexures; the geometry and the number of the flexures, and the size of prescribed motion gap(s) between the suspended fixation bodies and the outer frame.

One goal of the compliant bone plate disclosed herein is to allow axial motion, to resist torsion and bending, and to provide a compliant bone plate that will not fail within the body. In some embodiments, the axial motion can be a function of (i) the size of the gap between the suspended fixation body and the frame, (2) the flexures-their geometry, dimensions, and quantity. For example, if the flexure is linear, the axial motion can be a function of the flexure's length. The torsion and bending resistance can be a function of the thickness of the frame, the suspended fixation bodies, and the flexures. The compliant bone plate's failure can be a function of the number of cycles the flexures can handle before failure, which can be a function of the flexures' thicknesses. With this understanding, one of ordinary skill in the art will understand that the adjustable variables can be manipulated to design a compliant plate for a particular patient.

For example, as one of ordinary skill in the art will appreciate, the pseudo-rigid-body model can be used to predict the force- and stress-deflection response of various flexure topologies used in the plate. Thus, the flexures can be modeled using the PRBM to design a compliant bone plate for a particular patient.

As is explained in greater detail below, in some embodiments, the compliant bone plates described herein can be made from a single piece of material. Making the compliant bone plate from a single piece of material (e.g., a metal plate), can make it relatively simple and inexpensive to manufacture and simultaneously reduce the number of components that could detach and become lost in the body. Further, it can produce a compliant bone plate that does not wear, which is an advantage over compliant plates that favor axial motion through the use of sliding parts, silicone envelopes/inserts, or multiple materials/components that articulate along rigid surfaces.

In some embodiments, the compliant bone plate can be formed by removing material, e.g., the space between the frame and the suspended fixation bodies, from a single piece of material, e.g. a steel plate. For example, in some embodiments, the compliant bone plate can be formed by stamping a single sheet of metal. In another embodiment, the compliant bone plate can be formed by removing material from a single piece of material using a water jet, a laser, or wire electrical discharge machining (EDM), for example. Further, as one of ordinary skill in the art will appreciate, any manufacturing method known in the art can be used to create the compliant bone plate as disclosed herein, including, for example, additive manufacturing.

The compliant bone plates described herein can be made of many different biocompatible materials, as one of ordinary skill in the art will appreciate. Titanium and stainless steel are two example suitable materials. While titanium plates (e.g., titanium alloy, often Ti-6Al-4V) have gained popularity due to their reduced stiffness, they are generally more expensive than steel and their tissue ingrowth properties may not be desired since it can cause difficulty when removing implants. One advantage of the compliant plates described herein is they can be manufactured of stainless steel, which is often preferred by surgeons for these reasons, while still allowing an effective stiffness much lower than rigid stainless steel plates.

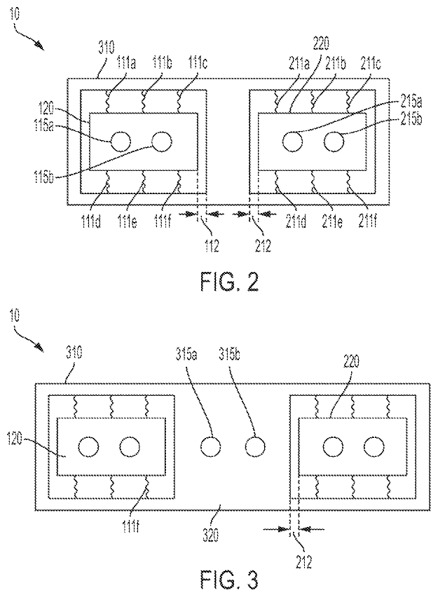

Applicant uses the terms “length,” “width,” and “thickness” herein to refer to certain features of the disclosed subject matter. With reference to FIG. 2, Applicant proposes a coordinate system in which the x-axis runs left-to right, the y-axis runs bottom to top, and the z-axis runs perpendicularly into the page. With continued reference to FIG. 2, the compliant bone plate's length is its measurement in the x-direction, its width is its measurement in the y-direction, and its thickness is its measurement in the z-direction.

Referring now to the drawings, in which like numerals represent like elements, examples of the present disclosure are herein described. FIG. 2 illustrates an embodiment of a compliant bone plate 10 having two suspended fixation bodies 120, 220, according to an embodiment of the disclosure. As shown in FIG. 2, a compliant bone plate 10 can comprise an outer frame 310, one or more suspended fixation bodies 120, 220 each comprising one or more screw holes 115a, 115b, 215a, 215b that can be used for fixing the suspended fixation bodies 120, 220 to the bone fragments with either locking or conventional screws, and compliant, or flexible, connections 111a-f, 211a-f (each, a “flexure”) between the outer frame 310 and the suspended fixation bodies 120, 220. In this configuration, the suspended fixation bodies 120, 220 can translate axially (for long bones, along the diaphyseal axis of the bone; for other anatomical sites, axially simply implies unidirectional across the fracture site) towards and away from one another. One intentional design feature that can be included is a prescribed gap 112, 212 between the suspended fixation bodies 120, 220 and the outer frame 310. Each gap 112, 212 limits the amount of motion that the respective suspended fixation body 120, 220 can translate axially relative to the outer frame 310 before buttressing up against the outer frame 310. By defining this gap 112, 212, the exact amount of interfragmentary motion can be controlled. As physiological loading is transferred from one bone fragment to the compliant bone plate 10, it can initiate the movement of its attached suspended fixation body towards the adjacent suspended fixation body.

The concept of a prescribed motion gap can apply for both primary and secondary healing. In secondary healing, the prescribed motion gap defines the range of motion that bone fragments are free to axially translate, which is known to directly influence bone healing. Too little or too much motion and fractures may not heal. For primary healing, it could still act as a safe stop preventing either too much compression or further expansion under tensile loads which could lose the compression needed for primary healing.

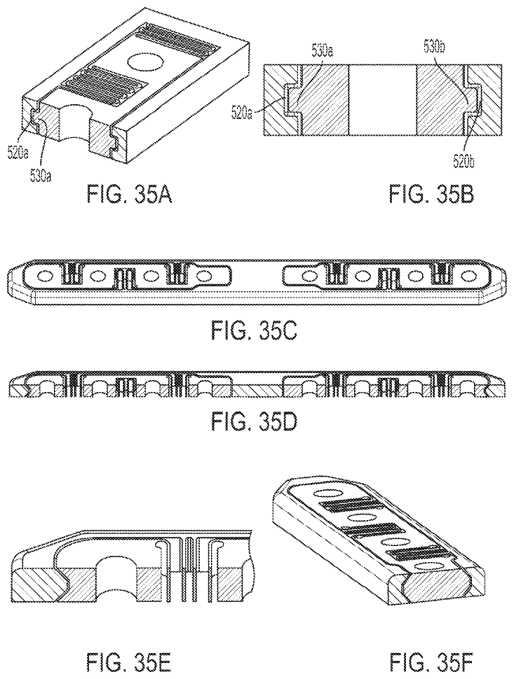

In some embodiments, two or more flexures are preferred to attach one suspended fixation body to a frame since this can change the boundary conditions and help guide the motion of the suspended fixation body to be axial, especially if there is one flexure on either side. However, single-flexure designs are possible. For example. In some embodiments, a single flexure (on the top or bottom) can allow for motion of the suspended fixation body, while an axial groove/protrusion in the outer frame/suspended fixation body (e.g., FIGS. 35A and 35B) guides the motion such that it remains axial.

In the embodiment illustrated in FIG. 2, the flexures 111 contact the suspended fixation bodies 120, 220 transversely (on the sides), not the top and bottom (main axis of device 10). This is important because it leverages the bending compliance of the flexures 111 instead of axial compression/tension of the flexures 111.

FIG. 3 illustrates an embodiment of a compliant bone plate 10 with screw holes 315 in the outer frame 310, according to an embodiment of the disclosure. This embodiment could be useful for the treatment of multi-fragmented bone fractures. In some embodiments, the compliant bone plate 10 can consist of more than two suspended fixation bodies, or the addition of screw holes 315a, 315b in the middle 320 of the outer frame 310. Such an embodiment can be useful for the treatment of multi-fragmented fractures. For example, a diaphyseal shaft fracture of a long bone consisting of one transverse fracture in the proximal portion of the shaft, and another in the distal portion of the shaft, could be treated with a compliant bone plate 10 that has screw holes 315a, 315b in the middle portion 320 of the outer frame 310. This could allow one or more screws to attach one suspended fixation body 120 to the proximal-most bone fragment, one suspended fixation body 210 to the distal-most bone fragment, and the middle portion 320 to a middle bone fragment.

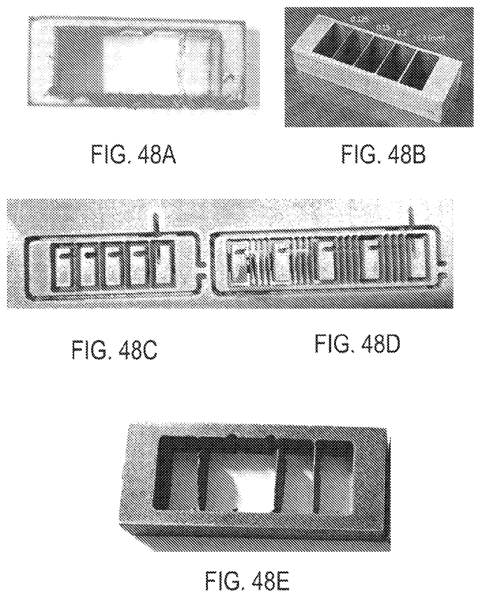

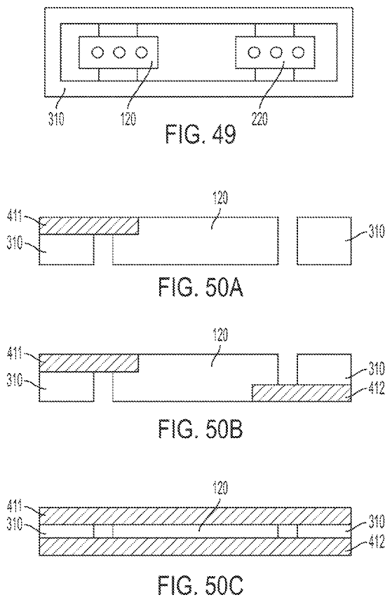

FIG. 48 shows a compliant bone plate with two suspended fixation bodies within a single hole in an outer frame, according to an embodiment of the present disclosure. In some embodiments, there is only one suspended fixation body in the frame. However, the subject matter disclosed herein is not so limited. Instead, a complaint bone plate could have multiple suspended fixation bodies axially moving towards one another within the same aperture in the frame, as illustrated in FIG. 48. In this case the motion would be limited by the distance between the two suspended fixation bodies instead of the suspended fixation bodies and the outer frame. Under certain conditions the performance could be quite similar whether there is a bridge in the middle of the frame creating two apertures or not.

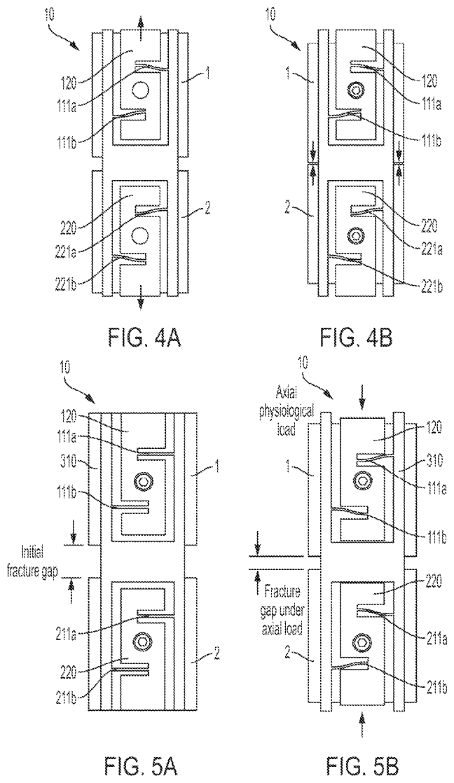

FIGS. 4A and 4B illustrate how a compliant bone plate 10 can be used to target primary healing, according to an embodiment of the disclosure. As illustrated in FIGS. 4A and 4B, in some embodiments, a compliant bone plate 10 can specifically target primary (direct) healing via complete anatomic reduction and interfragmentary compression. When compression at the fracture site, or across multiple fracture sites, is desired, the compliant bone plate 10 can leverage the principle of storing strain energy to provide compression. After anatomic reduction, the suspended fixation bodies 120, 220 can be pulled away from the fracture site such that the elastic flexures 111a, 111b, 211a, 211b are pre-strained, as illustrated in FIG. 4A. Then, the compliant bone plate 10 can be fixed to the bone 1, 2 on each side of the fracture with either locking or compression screws, and then the suspended fixation bodies 120, 220 can be released. Upon releasing the suspended fixation bodies 120, 220 the stored strain energy in the flexures 111a, 111b, 211a, 211b will cause a resultant compression force between bone fragments 1, 2, as illustrated in FIG. 4B. This compression could be maintained in vivo long after surgical implantation and can be more predictable than compression generated through traditional methods with eccentric placement of screws in compression plates. If the flexures 111a, 111b, 211a, 211b are designed such that the fatigue endurance limit stress value is not reached during intraoperative pre-strain or in response to expected physiological loading, the flexures 111a, 111b, 211a, 211b can avoid stress relaxation and creep phenomenon and can theoretically provide the compression force with infinite life.

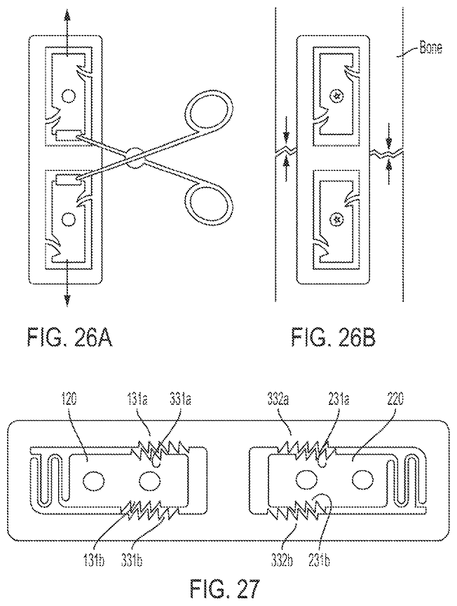

To provide another example of how a compliant bone plate 10 can be used to target primary healing, according to an embodiment of the disclosure, FIGS. 26A and 26B are provided. FIGS. 26A and 26B illustrate an example surgical instrument distracting a compliant bone plate into tension, then being released.

FIGS. 5A and 5B illustrate how a compliant bone plate can be used to target secondary healing, according to an embodiment of the disclosure. As illustrated in FIGS. 5A and 5B, in some embodiments, a compliant bone plate 10 can specifically target secondary (indirect) healing via callus formation due to interfragmentary strain. When dynamic, interfragmentary strain is desired at the fracture site, an axially compliant bone plate 10 can be fixed to the bone fragments 1, 2 in a similar manner as would be done with a standard, rigid metallic bone plate. Anatomic reduction can be performed, while maintaining some desired fracture gap (in some embodiments, this can be between 1 and 5 mm). Then, locking or conventional screws can be used to fix the compliant bone plate 10 to the bone 1, 2. In its resting state, the flexures 111a, 111b, 211a, 211b of the compliant bone plate 10 are not required to be pre-strained or store strain energy. When physiological loading occurs, the load is transferred from the bone 1, 2 to the screw(s), and from the screw(s) to the suspended fixation bodies 120, 220, causing axial motion of the suspended fixation bodies 120, 220 relatives to the outer frame 310. This can cause interfragmentary motion that is required for secondary healing via callus formation.

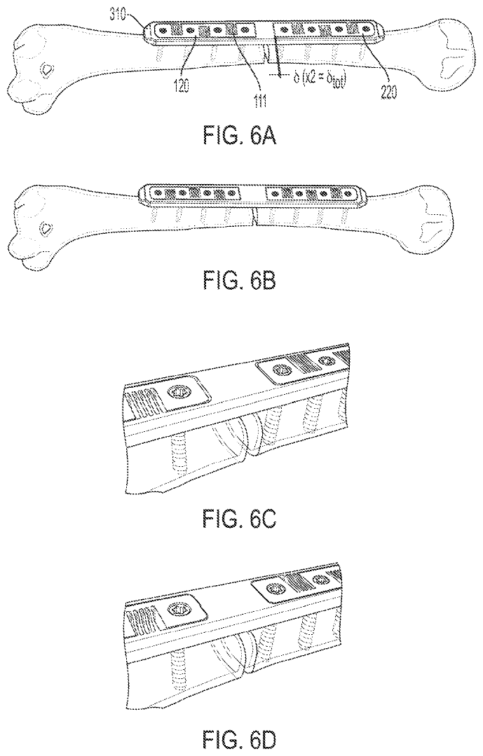

FIGS. 6A-6D illustrate a long bone fracture fixed with a flexure-based compliant bone plate 10, according to an embodiment of the disclosure. In FIGS. 6A-6D, a compliant bone plate 10 in accordance with the present disclosure is shown in its clinical scenario, fixed to a fractured bone. The top image shows an annotated plate 10 utilizing serpentine flexures 111. FIG. 6B shows a plate 10 utilizing straight flexures 111 in parallel. FIG. 6C shows the plate 10 in its undeflected state, when no axial load is being applied to the bone. FIG. 6D shows the plate 10 in its deflected state, when a sufficiently large axial load is applied to the bone, and thus the suspended bodies 120, 220 in the plate 10, resulting in the flexures 111 elastically deflecting until the prescribed motion gap closes. The plate 10 shown in FIGS. 6A-6D is designed such that axial motion is allowed only until the prescribed motion gap 112, 212 closes and the suspended bodies 220, 120 bottom out and contact the outer frame 310. This is done to prevent over-strain of the fracture site. The target range of allowed axial motion is typically targeted to be between 0.3 and 2 mm. The range of motion that the plate 10 allows is the sum of the prescribed motion gaps 112, 212 present in the plate 10.

In the embodiment illustrated in FIGS. 6A-6D, there are multiple (four) screw holes per suspended fixation body 120, 220. By having multiple holes per suspended fixation body, many different groups of flexures 111 can attach to that suspended fixation body 120, 220 at locations far from one another increasing the bending and torsional stiffness. Further, having multiple screw holes per suspended fixation body 120, 220 ensures that all flexures 111 are engaged and contribute to axial stiffness, regardless of whether all holes are used.

In addition, in the embodiment illustrated in FIGS. 6A-6D, all screw holes are suspended, meaning that they are formed through a suspended fixation body 120, 220. So instead of a suspended hole (i.e., a hole in a suspended fixation body) moving towards and away from a fixed hole (i.e., a hole not in a suspended fixation body), there are no fixed holes and instead two groups of suspended holes moving towards and away from one another. This is done for performance reasons; it allows the total interfragmentary motion to be shared between both sides of the plate instead of just one, reducing stresses in the flexures.

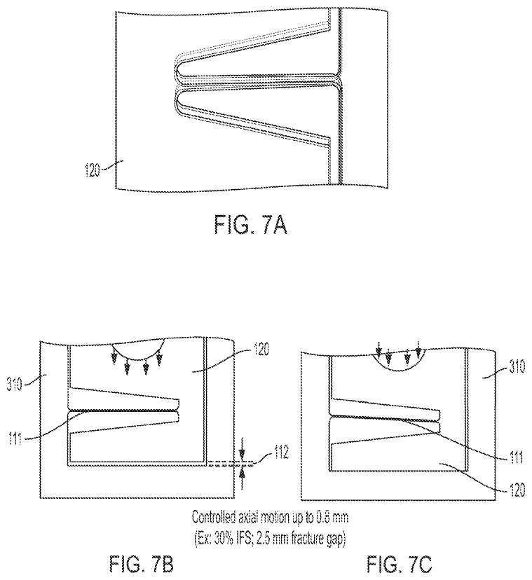

FIGS. 7A-7C illustrate how a straight flexure 111 can undergo axial deflection, according to an embodiment of the disclosure. As used herein, “axial” refers to the x-axis as is defined earlier (main axis of the plate/bone). In FIGS. 7A-7C, a straight flexure 111 is shown connecting the outer frame 310 to the suspended body 120. This flexure 111 is known as is a fixed-clamped flexure when such boundary conditions are applied (fixed on one end, purely vertical deflection allowed on the other end). In some embodiments, a compliant bone plate 10 can consist of a plurality of these straight flexures 111, which could allow the axial movement of the screw holes relative to the outer frame 310. Fixed-clamped flexures 111 possess stress-stiffening effects and can prevent transverse motion when paired in a symmetrical or alternating configuration (e.g., flexures on either side of the point of loading). As more load is transferred to the bone and the straight flexures 111 continue to deflect, as illustrated in FIG. 7B, they become stiffer and require more force per unit deflection, which can prevent over-strain. This can be desirable for allowing the bone to experience relatively large amounts of interfragmentary strain (required for callus formation) in the presence of small physiological loads, while becoming stiffer and working to prevent over-strain under larger physiological loads.

In the embodiment shown in FIGS. 6A and 6B, the prescribed axial motion is 0.8 mm, which is determined by the size of the prescribed gap 112. However, as one of ordinary skill in the art will appreciate, the subject matter disclosed herein is not so limited. Rather, other amounts of motions can be allowed based on the design of the compliant bone plate (e.g., the size of the prescribed gap 112).

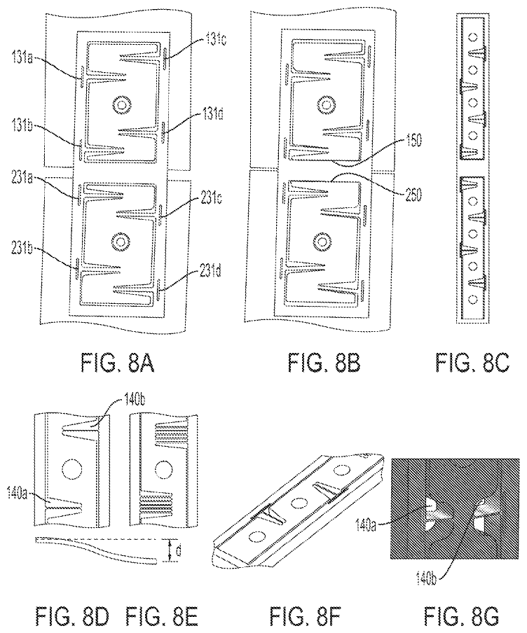

FIGS. 8A-8G illustrate various embodiments of compliant bone plates with straight flexures, according to embodiments of the disclosure. As shown in FIGS. 8A-8G, the suspended fixation bodies can axially translate towards one another due to the deflection of straight flexures that connect the suspended fixation bodies to the outer frame. In simulating the stresses present in straight flexure during actuation, it is observed that large stresses can occur even at small deflections. To compensate for this nonlinear stress profile of straight flexures, stress-relief slots 131a-d, 231a-d can be designed or machined into the static outer frame and/or suspended fixation bodies. To reduce stresses present in the flexures during deflection, the stress-relief slots 131a-d, 231a-d can allow the end of the respective straight flexure to translate and rotate slightly relative to the outer frame. This can reduce the maximum stress present in the compliant bone plate during loading while still facilitating the same amount of interfragmentary motion.

The embodiments shown in FIGS. 8A-8G are single piece mechanisms and can be constructed with subtractive manufacturing techniques such as wire electrical discharge machining (EDM), laser cutting, water jetting, or CNC milling, among others starting from a single piece of material. By having the potential to be made from a single piece, the manufacturing and assembly can be considerably less strenuous, expensive, and prone to error. However, the subject matter disclosed herein is not so limited. Instead, as one of ordinary skill in the art will appreciate, the compliant bone plate can be constructed by any manufacturing technique known in the art, including, for example, additive manufacturing.

Further, the embodiments in FIGS. 8A-8G can reduce or eliminate wear. Instead of achieving motion by the sliding of inserts within slots in a plate, for example, the present disclosure can achieve similar axial motion while reducing the friction and interaction between components. In the embodiments shown in FIGS. 8A-8G, the only dynamic interaction between solid surfaces that is expected to occur is the bottoming out of the flat surfaces on the fracture-facing portion of the suspended fixation bodies against the flat surfaces of the outer frame, at 150 and 250. However, this is expected to be a primarily pressing motion as opposed to a sliding motion under expected physiological axial loading.

In the embodiments shown in FIGS. 8A-8G, each suspended fixation body can comprise centralized screw holes at a uniform and common spacing (in some embodiments, generally around 18 mm for large, long bone shafts, smaller for other anatomical sites). In addition, in some embodiments, the suspended fixation bodies can possess non-rectangular profiles with cut-out features 140a, 140b extending inwards from the outer surface of the suspended fixation body (where it nears the outer frame). This can allow for the use of a longer flexure. From mathematical modeling, simulations, and experimentation on straight flexures, it is understood that increasing the flexure's length can be advantageous for certain compliant bone plate embodiments. For example, increasing flexure length can allow a flexure 111 to be thicker, stiffer, and able to deflect a larger amount of vertical deflection while maintaining a lower maximum stress. In some embodiments, these cut-out features 140a, 140b can be located in between screw holes to allow for longer cut-out features 140a, 140b. However, as one of ordinary skill in the art will appreciate, the subject matter disclosed herein is not so limited. Instead, the cut-out features 140a, 140b may be in-line with screw holes.

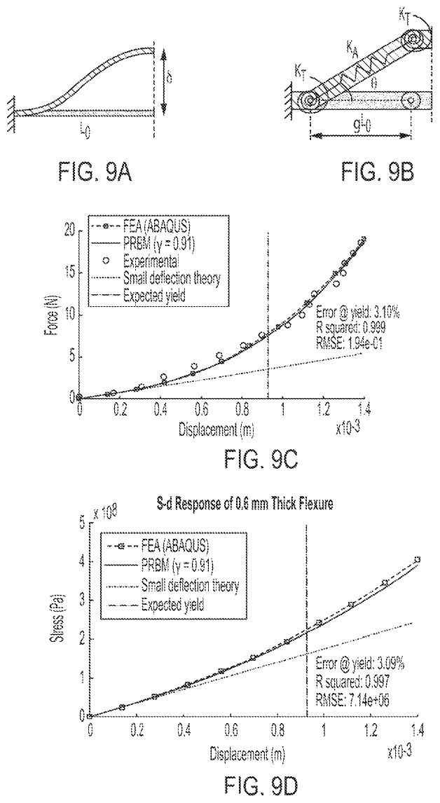

FIGS. 9A-9D are graphs that model the displacement of straight flexures, according to an embodiment of the disclosure. As illustrated in FIGS. 8A-8D, the displacement of straight flexures can be modeled with mathematic equations to determine optimal geometric parameters. In other words, mathematical modeling techniques can be used to predict the force- and stress-deflection response for flexures undergoing axial loading. This can be used in the present orthopedic application to predict the force and stress response for a compliant bone plate consisting of such flexures. The modeling technique employed in FIGS. 8A-8D is known as the pseudo-rigid-body model, approximating flexures as a series of rigid members connected by pins and torsional springs.

Shown in FIG. 9A, a fixed-clamped flexure is a flexure subjected to a vertical applied force such that the clamped end must move vertically along its boundary with no horizontal translation or rotation. The pseudo-rigid-body model for this flexure type is also shown in FIG. 9B. Fixed-clamped flexures are selected for use in the compliant bone plate due to their stress-stiffening effect and small profile, allowing them to provide adequately large stiffness and to be stacked efficiently.

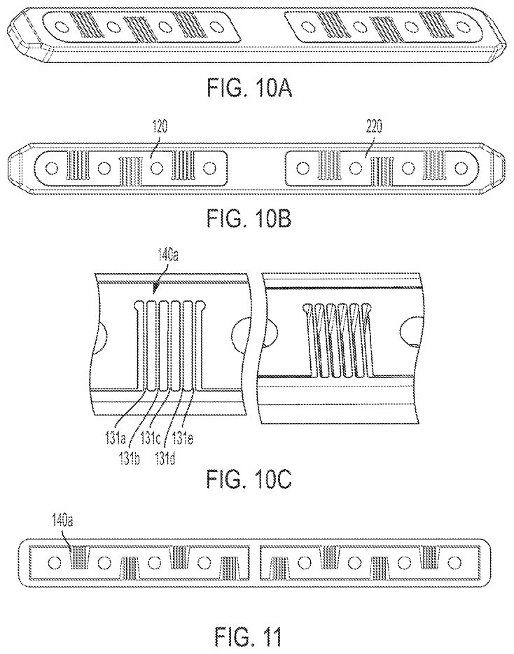

FIGS. 10A-10C and 11 illustrate various embodiments of compliant bone plates having a plurality of straight flexures in parallel, according to embodiments of the disclosure. As illustrated in FIGS. 10A-10C and 11, in some embodiments, each suspended fixation body can be connected to the outer frame by many straight flexures, including multiple straight flexures 131a-e within cut-out features 140a of the suspended fixation body. By increasing the number of flexures present in this manner (i.e., using a plurality of straight flexures in parallel on one side of the compliant bone plate), the total stiffness of the compliant bone plate can be increased and fine-tuned, while maintaining the same maximum stress present in each flexure during motion.

As illustrated in FIGS. 10A-10C and 11, screw holes in the plates can be in-line with one another through the mid-line of the plate, or off-set from the mid-line of the plate, or staggered such that screw hole distance from plate midline varies between holes (for example, alternating offset screw holes). Screw holes can also be angled in alternating directions such that screw hole axis is not perpendicular to plate but rather angled so as to direct the screws into the bone at different angles for different screw holes.

In some embodiments, as illustrated in FIG. 10B, the flexures on one suspended fixation body 120 are the same as or a mirror image of the flexures on the other suspended fixation body 220. However, as one of ordinary skill in the art will appreciate, the subject matter disclosed herein is not so limited. Instead, the flexures on one suspended fixation body can be different than the flexures on the other suspended fixation body. In such an embodiment, the flexures can have different stiffnesses, which can cause one of the suspended fixation bodies to axially translate before the other suspended fixation body.

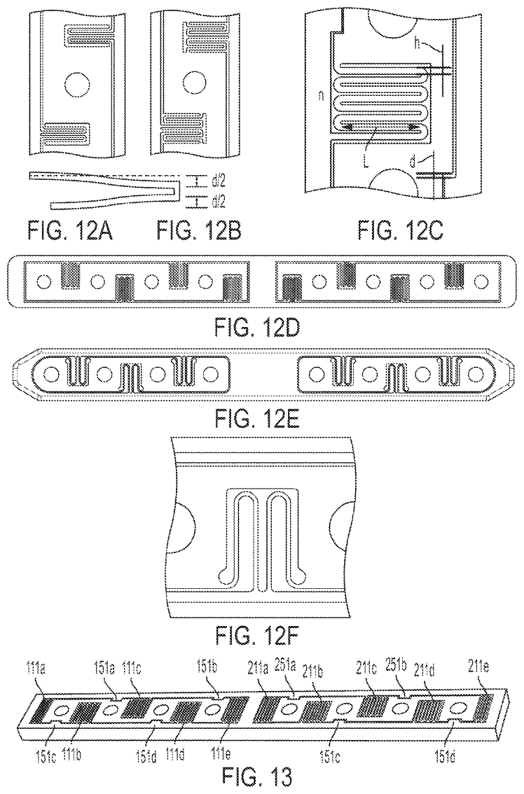

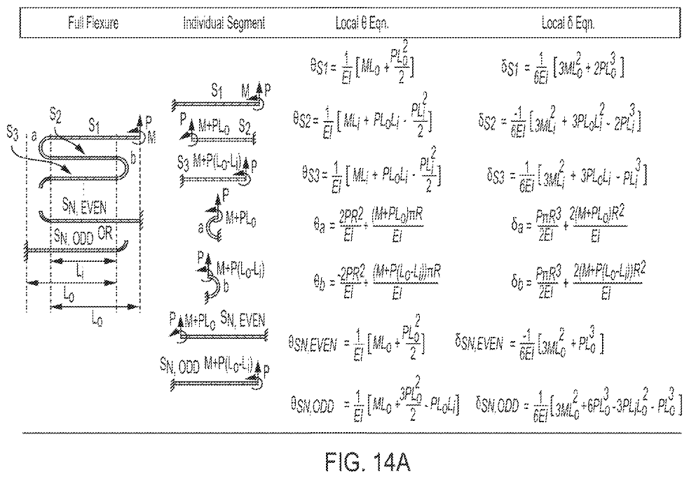

FIGS. 12A-12F and 13 illustrate various embodiments of compliant bone plates with switchback flexures, according to embodiments of the disclosure. As illustrated in FIGS. 12A-12E and 13, in some embodiments, the suspended fixation bodies can be connected to the outer frame with switchback flexures 111, named after their resemblance to windy paths or roads up a steep natural incline. These types of flexures can also be referred to as serpentine flexures. The number of turns present defining the total number of straight segments in each switchback can vary depending on the target stiffness and stress. When more turns are present, the total amount of deflection that each straight segment must deflect decreases. This amount of vertical deflection per straight segment is approximately equal to the total prescribed vertical deflection divided by the number of straight segments per compliant element. Compliant units can include a multiple of uniform or varying compliant elements.

As used herein, the terms “compliant element” and “compliant unit” is used to differentiate between individual flexures (each, a complaint element) and groups of flexures (each, a compliant unit). For example, in one cut-out window of the suspended fixation body, there could be one compliant element (i.e. a serpentine flexure with many turns) or a compliant unit consisting of many compliant elements (i.e. many straight flexures in parallel).

FIG. 13 illustrates an embodiment of a compliant bone plate having step-like features and switchback flexures, according to an embodiment of the disclosure. As shown in FIG. 13, in addition to flexures being present along the lateral sides of the suspended fixation bodies and in between screw holes (as illustrated in FIG. 12D), flexures can be present at the top and bottom of the suspended fixation bodies (111a, 111e, 211a, 211e) to form additional connections between the suspended fixation bodies and the outer frame, thus increasing the axial stiffness and improving the resistance to bending and torsion by increasing the number of connections through which loads can be transferred from the suspended fixation bodies to the outer frame. In such embodiments where compliant elements are present centrally at the top and bottom of the suspended bodies, the contact feature between suspended bodies and outer frame which controls and limits the amount of total axial motion can be moved to the sides of the suspended fixation bodies and can take the form of one or multiple arranged step-like features 151a-d, 251a-d. In other words, the gap that defines the amount of interfragmentary motion in embodiments with upper and lower flexures can be located at the step-like features 151a-d, 251a-d.

In some embodiments, a compliant bone plate can include multiple types of flexures to guide the axial motion. For example, a compliant plate can include straight (111b-d, 211b-d) and switchback flexures (111a, 111e, 211a, 211e), as shown in the embodiment of FIG. 11. Such a combination allows for the benefits of each type of flexure to be provided to the compliant bone plate. In this scenario, the straight flexures can help to prevent transverse motion and undesired torsion and bending due to their short working length and increased off-axis stiffnesses; the switchback flexures can allow for greater axial stiffness to be achieved since the thickness of the flexures can be thicker and deflection is shared between each straight portion within each switchback element.

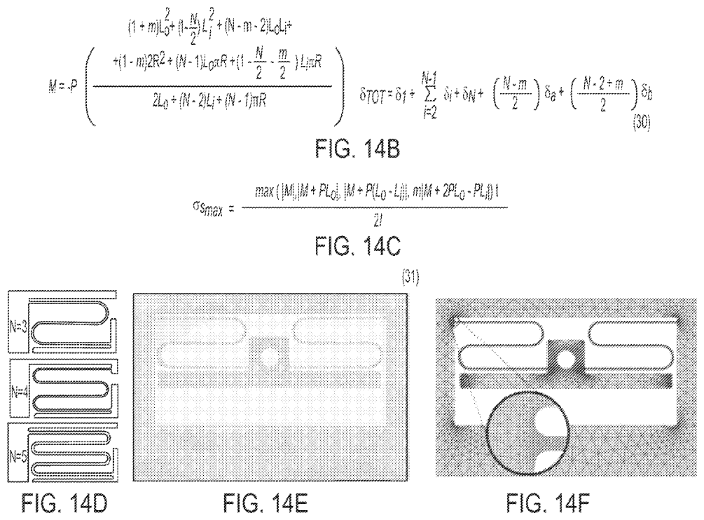

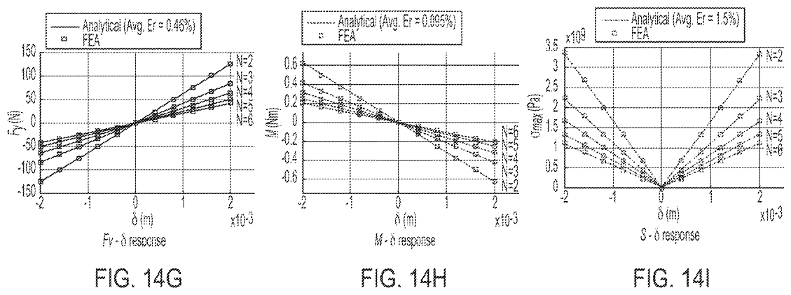

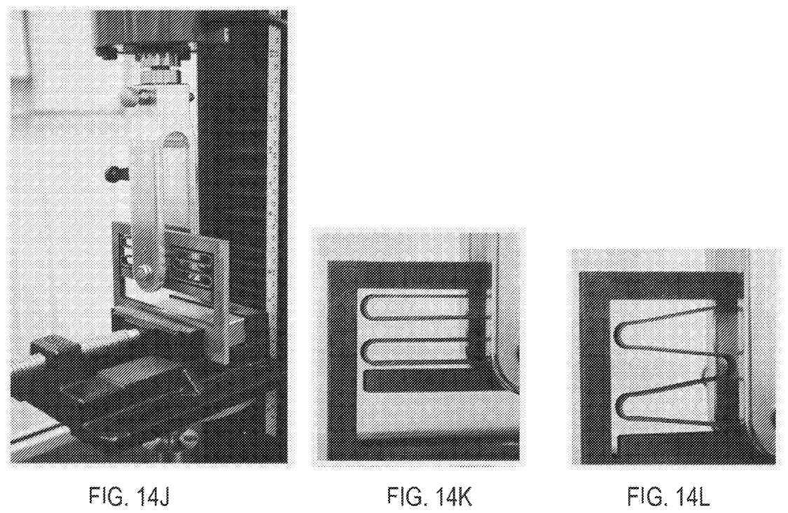

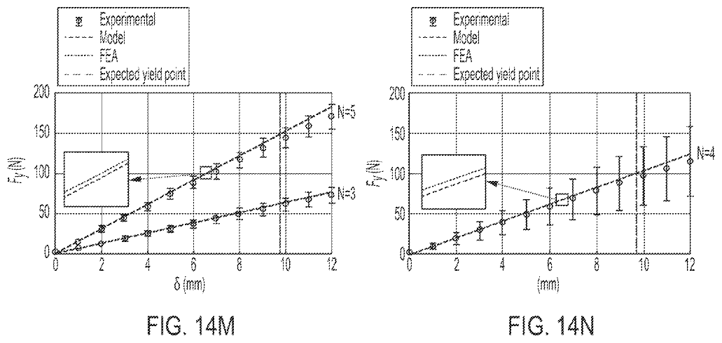

FIGS. 14A-14N illustrate how serpentine flexures can be modeled with mathematic equations to determine optimal geometric parameters, according to an embodiment of the disclosure. Mathematical modeling methods can be used to understand and predict the performance of serpentine flexures for use in the present compliant bone plate. Our analytical models have been shown to be accurate within ˜5% for predicting the stiffness of serpentine flexures compared to simulations and experimental testing. The models can also predict the maximum location and magnitude of stress in the serpentine flexures based on an input force or deflection load and the geometrical and material property parameters of the flexures. This is useful since it allows the designer to select appropriate flexure thickness, number of segments in the serpentine, radius of semi-circle segments, etc. without exceeding a maximum allowable stress for a required vertical deflection.

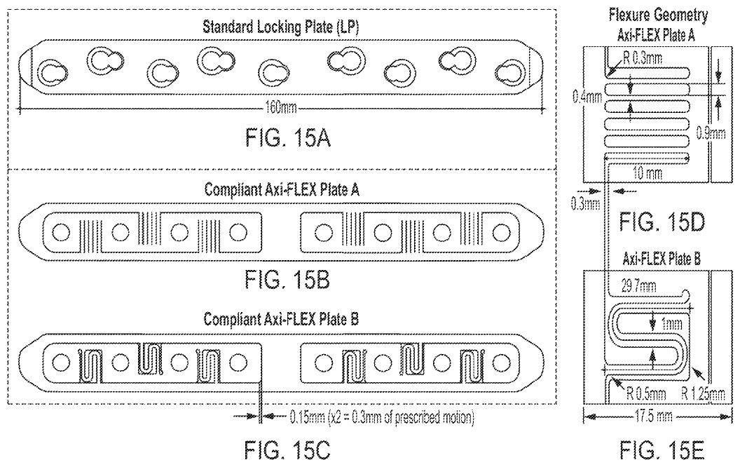

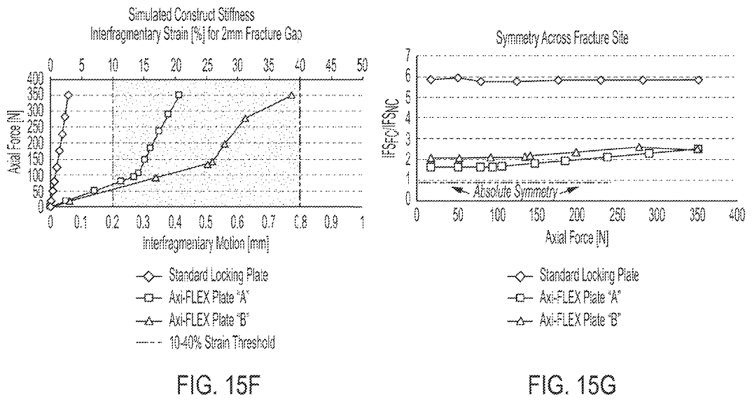

FIGS. 15A-15G show example dimensions and axial loading simulation results for the present disclosure consisting of straight flexures or serpentine flexures with a target axial range of motion of 0.3 mm, according to embodiments of the present disclosure.

Two example embodiments of the compliant plates are shown, each targeting an axial range of motion of 0.3 mm (0.15 mm motion per side of the plate). This desired range of motion can be tuned for various clinical scenarios and the flexure geometry and prescribed motion gap can be adjusted accordingly. The compliant plate's dimensions are shown relative to a standard commercially available locking plate (FIG. 15A).

FIG. 15F shows axial stiffness results for the proposed compliant plates versus standard locking plates. The compliant plates exhibit a bi-phasic stiffness consisting of a flexible phase (when the flexures are free to elastically deflect) and a rigid phase (once the suspended bodies bottom out against the outer frame). This is desirable clinically since it delivers the minimum required interfragmentary motion under relatively small axial loads, while then becoming stiff in order to prevent over-strain, maintaining a desired interfragmentary strain under a large range of axial loads.

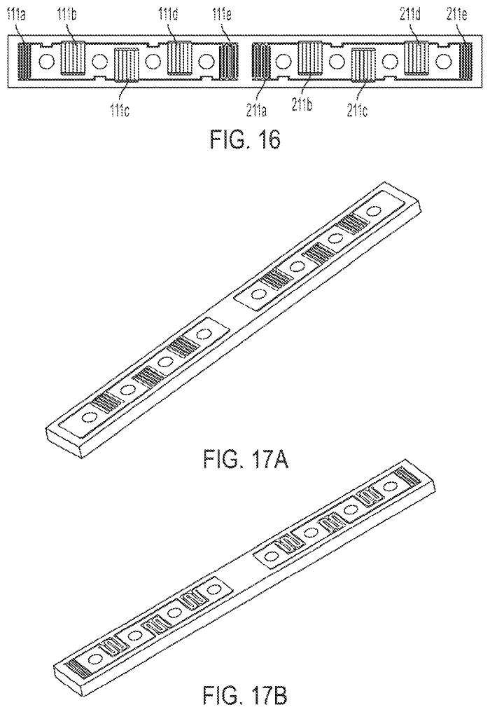

FIG. 16G shows interfragmentary motion symmetry results with the present disclosure versus a standard locking plate. A value of 1 represents identical interfragmentary motion at the near and far cortex, which experimental literature has shown to be most desirable for optimal bone healing. The compliant plates deliver an average symmetry ratio (far cortex micromotion vs near cortex micromotion) of 2.04, compared to a symmetry ratio of 5.85 for the traditional locking plate. This illustrates the pure axial motion advantages of the present disclosure; while traditional locking plates suppress motion between bone fragments and resort to bending and asymmetrical micromotion, the compliant plate designs can deliver greater magnitude and symmetry of motion which is known to facilitate improved secondary healing.

FIG. 16 illustrates a compliant bone plate with multiple types of compliant elements, according to an embodiment of the disclosure. In FIG. 16, an example embodiment is shown which has both straight and switchback compliant elements present to guide the axial motion. For example, a compliant plate can include straight (111b-d, 211b-d) and switchback flexures (111a, 111e, 211a, 211e), as shown in the embodiment of FIG. 16.

FIGS. 17A and 17B illustrate various embodiments of compliant bone plates having flexures with an increased thickness, according to embodiments of the disclosure. The maximum thickness of the flexures can be determined by the desired amount of interfragmentary motion. When large motion is desired, stresses can rise in the flexures during deflection, and thus thinner flexures can be required since the maximum stress is correlated to the thickness. When the amount of interfragmentary motion desired decreases, flexures can be thicker, as illustrated in FIGS. 12A and 12B. Thicker flexures can be easier to manufacture, have increased relative tolerance, and have increased stiffness per flexure unit, requiring fewer to achieve the desired stiffness.

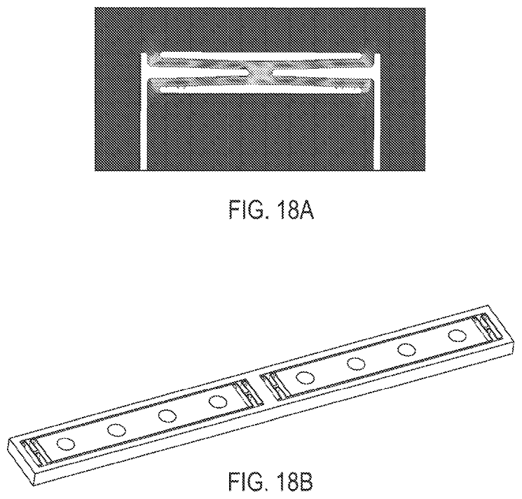

FIGS. 18A and 18B illustrate various views of an embodiment of a compliant bone plate having lamina emergent torsional (LET) joint flexures, according to an embodiment of the disclosure. FIGS. 18A and 18B show that compliant elements can also consist of one or LET joints. While these are typically used for aiding in the out-of-plane motion of a mechanism, placing LET joints at multiple planar locations within the compliant bone plate, such as at the top and bottom of each suspended fixation body, can facilitate axial motion while preventing out-of-plane motion.

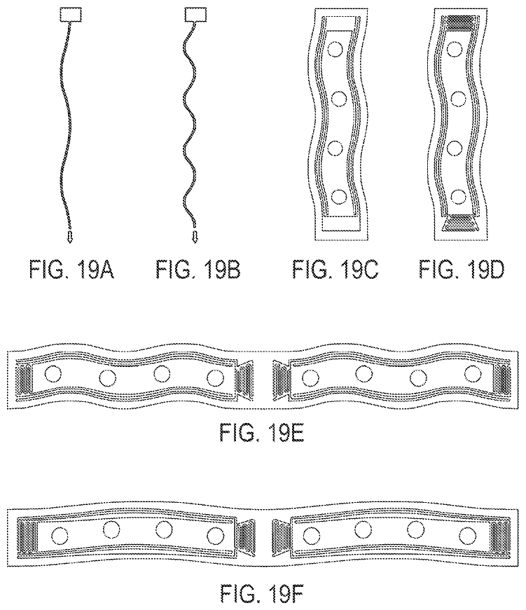

FIGS. 19A-19F illustrate various embodiments of compliant bone plates having curved-member flexures to be elongated, according to embodiments of the disclosure. In some embodiments, one or more suspended fixation bodies can be attached to the outer frame with curved-member flexures 111. A curved-member flexure 111 can include one or more curves or humps. The curved-member flexure 111 can be elongated in order to guide axial motion of the suspended bodies relative to the outer frame.

FIGS. 19A-19F include examples of singular flexures that are curved which can be elongated with a vertical force. FIGS. 19C and 19D show some example half-plate and FIGS. 19E and 19F show some example full-plate embodiments. Each of the embodiments in FIGS. 19C-19F incorporate curved flexures and cutouts along the flexures with enough clearance (negative space) on either side of the curved members to ensure that the flexure is allowed to deflect and contact, and wear does not occur during deflection. Because the effective length of the flexure can be much longer when it is allowed to run vertically along the full length of one half of the plate, long curved members to be elongated have the potential of requiring a larger force to achieve the same axial deflection. This can be advantageous when designing for a plate with increased stiffness.

In some embodiments, the axial movement of suspended fixation body can be limited only by the straight length of one of the flexures. In other embodiments, it can be limited by the distance between the suspended fixation body and the outer frame (i.e. the flexures starts to straighten out but before fully straightened, contact is made between suspended fixation body and frame, limiting motion).

Further, FIGS. 16D-16F show an important and strategic combination of long, curved-member flexures to add stiffness to the system and smaller switchback units at the top and bottom of each suspended fixation body to add stability. The long, curved members may not be desirable by themselves since they only have two connection points to the outer frame and such a long effective length, and thus will have poor resistance to bending and torsion. However, augmenting such a compliant bone plate having curved-member flexures design with other compliant units connecting the same suspended fixation body to the outer frame at different points can improve the bending and torsional rigidity of the compliant bone plate.

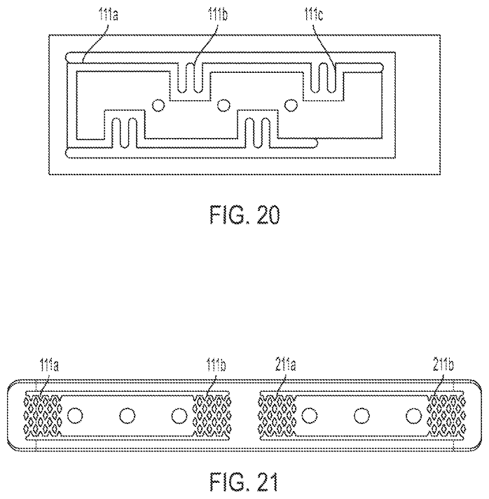

FIG. 20 illustrates an embodiment of a compliant bone plate having long, segmented flexures 111a with switchback elements 111b, 111c in series, according to an embodiment of the disclosure. FIG. 15 illustrates how the stiffness of a compliant bone plate can be further increased by incorporating long flexures 111a with winding switchback flexures 111b, 111c in series. In this manner, the deflection can be spread out between each grouping of switchback flexures 111b, 111c, which can result in less deflection per group of switchback flexures 111b, 111c, reducing the stress due to deformation experienced in each element. This can allow for increasing the thickness of the flexures, increasing feasibility of manufacturing, and increasing the total axial stiffness of the compliant bone plate.

FIG. 21 illustrates an embodiment of a compliant bone plate having lattice- or polygon-inspired patterns, according to an embodiment of the disclosure. As illustrated in FIG. 21, in some embodiments, flexures connecting the outer frame to the suspended fixation bodies can take the form of lattices or patterns 111a, 111b, 211a, 211b created by defining the negative space as pattern polygons (diamonds, squares, pentagons, hexagons, etc.). Hexagons are a common patterned polygon found in nature, such as honeycomb.

In some embodiments, meso-material is used instead of flexures to carry the deflection. A meso-material can be composed of small unit cells that are patterned and connected resulting in a certain behavior of the plate at a macro level. In some embodiments, the pattern can be diamond-shaped, similar to the flexures 111a-111d shown in FIG. 16, or hexagonal, for example and not limitation. When such embodiments are compressed, the hexagons can flatten and the deflection can be distributed across some or all of the pattern. In some embodiments, the compliant bone plate can include meso-material at the middle of the compliant bone plate. In such embodiments, the hexagons (or other pattern) can be layered such that when the fixation body is loaded the hexagonal can be “squashed” down. Further, in some embodiments “S”-shaped pieces can be used instead of hexagons.

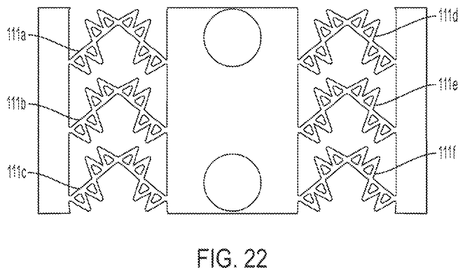

FIG. 22 illustrates an embodiment of a compliant bone plate having triangular-feature flexures, according to an embodiment of the disclosure. In some embodiments, a compliant bone plate can have triangular-feature flexures 111a-f. Triangular-feature flexures can have superior torsional rigidity, specifically with a high torsional stiffness to bending stiffness ratio. It can be possible to use planar, single-piece compliant bone plate designs that incorporate flexures with a large torsional stiffness-to-bending-stiffness ratio. It is desired for the flexures to bend in-lane, creating the axial motion of the compliant bone plate while resisting torsion, which causes bending of the compliant bone plate. For example, flexures with structural hollow features can allow bending while providing substantial resistance to torsion. The compliant bone plate of FIG. 22 comprises a flexure with triangular flexures 111a-f for torsion resistance. The compliant bone plate of FIG. 22 illustrates how the suspended fixation bodies can be connected to the outer frame with such flexures that can significantly improve the torsional stiffness of the flexures.

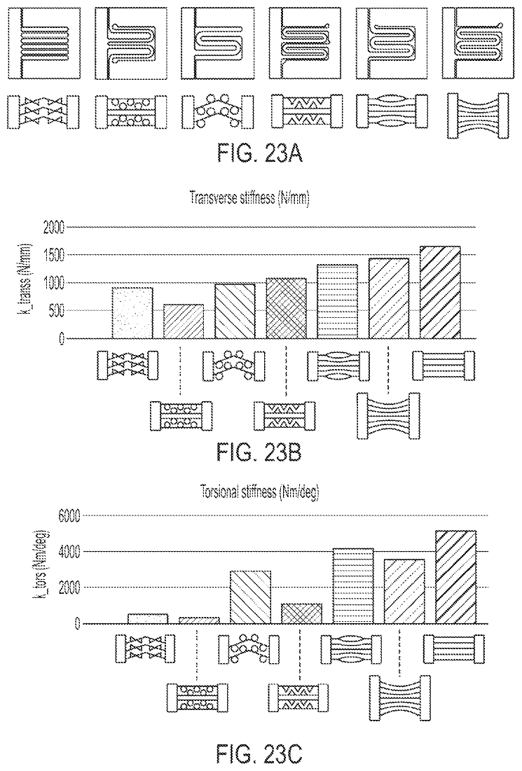

FIGS. 23A-23C illustrate the various flexures and their transverse and torsional stiffness, according to embodiments of the present disclosure. It is also possible to use planar, single-piece designs that incorporate flexures with a very large torsional and transverse stiffness. It is desired for the flexures to bend in plane, creating the axial motion of the plate, while resisting torsion, which causes bending of the plate. For example, flexures with structural hollow features can still allow bending while providing substantial resistance to torsion. The flexure topology candidates in FIG. 23A are all designed to be “stress-equivalent” in that they will exhibit approximately equal maximum stresses when subjected to the same prescribed vertical deflection. By comparing the transverse and torsional stiffness of different flexures, it becomes evident that straight flexures in parallel may offer the greatest off axis stiffness. However, some of the other flexures could offer advantages if iterated upon with respect to stacking or nesting flexure elements efficiently and possessing a greater off-axis stiffness per amount of flexure thickness. Each of these flexure topology candidates in FIG. 23A can be used to connect the outer frame to the suspended bodies.

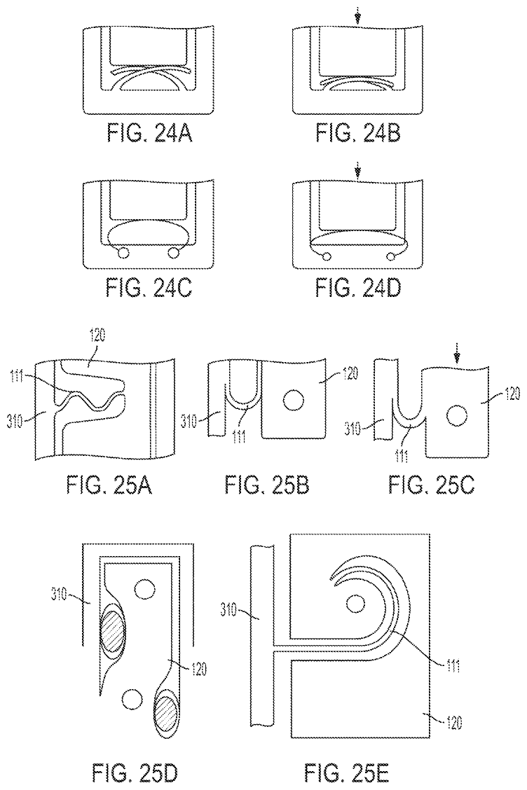

FIGS. 24A-24D illustrate flexures having fixed-free and pinned-pinned boundary conditions, according to embodiments of the disclosure. Fixed-free or pinned-pinned boundary conditions can allow for greater deflections and lower stress values during loading. To tailor the stiffness of the suspended fixation bodies relative to the outer frame, additional flexures with other boundary conditions can be incorporated. For example, fixed-free (cantilever) (as shown in FIGS. 24A and 24B) and pinned-pinned (as shown in FIGS. 24C and 24D) flexures that return lower stress values per amount deflection can be incorporated. This can be advantageous for making flexures thicker and tuning the axial stiffness without resulting in excessively large stresses.

FIGS. 25A-25E illustrate embodiments of compliant bone plates having pre-curved flexures, according to embodiments of the disclosure. As illustrated in FIGS. 25A and 25B, in some embodiments, the suspended fixation bodies can be connected to the outer frame with non-straight flexures. In one example, an “unrolling” flexure is incorporated with a “U” shape, as illustrated in FIGS. 25B and 25C. In another example, as shown in FIG. 25D, a rolling contact element can be incorporated inside of an oval shaped flexure group. This insert can prevent the transverse (horizontal) motion of the suspended fixture body relative to the outer frame.

FIGS. 26A and 26B illustrate a method of using a compliant bone plate according to the embodiments described herein to facilitate primary healing, according to an embodiment of the disclosure. FIGS. 26A and 26B illustrate that, in an embodiment, a compliant bone plate as described herein can be pulled into tension, affixed to bone fragments, and then released to create compression at the fracture site as the pre-strained flexures force the suspended fixation bodies back towards one another (resting configuration). Any of the embodiments disclosed herein can be adapted to be pulled into tension. In some embodiments, instrumentation can be used to aid in the pulling apart of suspended bodies. In other embodiments, inserts can be pre-inserted and then removed.

FIG. 27 illustrates an embodiment of a compliant bone plate having ratcheting features 131a, 131b, 231a, 231b, 331a, 331b, 332a, 332b for maintaining compression, according to an embodiment of the disclosure. In some embodiments, primary healing can also be achieved by using compliant elements to drive the axial compression and ratcheting features 131a, 131b, 231a, 231b, 331a, 331b, 332a, 332b to preserve the compression and prevent bone fragments from pulling apart. In some embodiments, the ratcheting features 131a, 131b, 231a, 231b, 331a, 331b, 332a, 332b, which can also be referred to as “mated teeth,” can bend slightly as the suspended fixation bodies 120, 220 are driven towards one another. One pair of teeth (e.g., 131a) can move past another pair of teeth (e.g. 331a) to lock the teeth in place, much like a zip-tie. In this way, the ratcheting features 131a, 131b, 231a, 231b, 331a, 331b, 332a, 332b can allow the suspended fixation bodies 120, 220 to translate toward one another, causing interfragmentary compression, but do not allow the reverse such that once ratcheting is complete, compression can be maintained.

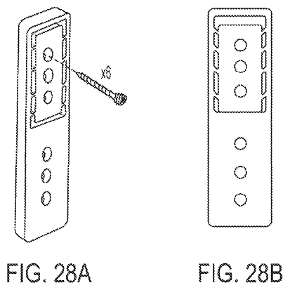

FIGS. 28A and 28B illustrate various views of an embodiment of an asymmetrical compliant bone plate, according to an embodiment of the disclosure. In some embodiments, axially compliant bone plates can also be designed and manufactured to have flexures on one side of the compliant bone plate only, as illustrated in FIGS. 28A and 28B. In this manner, the suspended fixation body can translate towards the static distal screw holes creating the dynamic compression for secondary healing or permanent compression for primary healing. This can simplify the manufacturing process since fewer flexures can be required. It can also double the axial stiffness of the compliant bone plate since the proximal and distal flexures normally act in series, halving the stiffness. This will, however, increase the maximum stress present in the flexures for the same amount of axial deflection, for the following reason: When a compliant bone plate with flexures on only one suspended fixation body (“asymmetrical compliant bone plate”) and a compliant bone plate with flexures on each suspended fixation body (“symmetrical compliant bone plate”) undergo the same axial deflection, the flexures in the asymmetrical embodiment will experience greater stress because the deflection is not shared across multiple flexures in series; rather, each flexure must undergo full axial deflection.

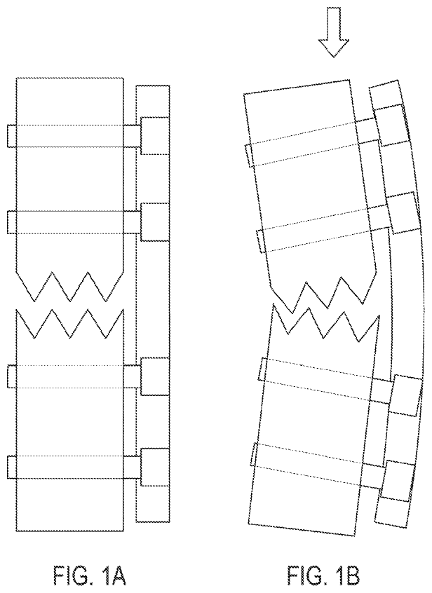

In some embodiments, a compliant bone plate may comprise bending and torsion rigidity reinforcements. There can be many instances where a compliant plate with a large bending stiffness and torsional stiffness is required. The embodiments described above can achieve sufficient performance in these categories depending on the geometrical parameters selected for the flexures and the desired amount of axial motion. The single-piece planar embodiments also allow for simplified top-down subtractive manufacturing and can facilitate axial motion without wear between components. In this section, additional features that can reduce the out of plane motion of the suspended fixation bodies relative to the outer frame (this is what causes the angular displacement of the plate under bending loads, as illustrated in FIGS. 1A and 1B) are presented.

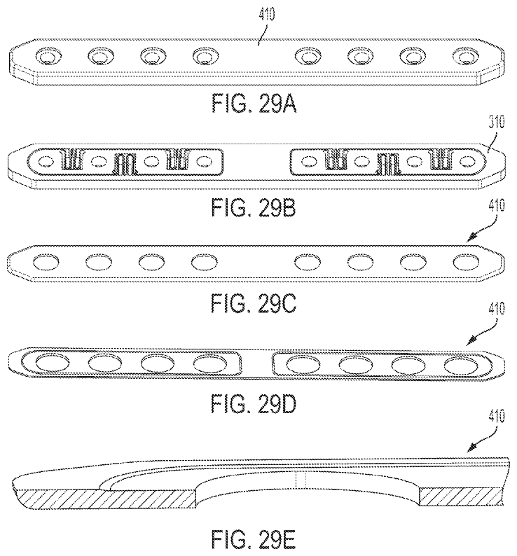

FIGS. 29A-29E show how, a rigid, thin support plate 410 can be added via pins, welding, or other manufacturing techniques or be designed directly into the static outer frame 310, according to an embodiment of the disclosure. This results in one or both sides of the compliant bone plate having such a thin support plate 410 on the outside. When the thin support plate 410 is connected to the outer frame 310, it adds rotational and bending stability to the outer frame 310 as well as ‘blocking’ or preventing the suspended fixation bodies from rotating out of plane relative to the outer frame 310. In some embodiments, the support plate 410 does not connect directly to the screws or the suspended bodies, allowing them to freely translate axially (the intended direction of motion) without interference from the support plate 410. However, when large enough off-axis loads are present and the suspended fixation bodies begin to rotate (caused by the torsion of the flexures), the support plate 410 can prevent the excessive rotation.

In FIGS. 29A-29E, a single support plate 410 is shown which has a uniform thickness except for a small, recessed feature on the face of the support plate 410 that faces the bone plate (see FIG. 29D). This results in a small gap between the support plate 410 and the suspended bodies 120, 220 and flexures 111 of the bone plate 10 such that translation can occur of the suspended bodies 120, 220 without interference with the support plate 410.

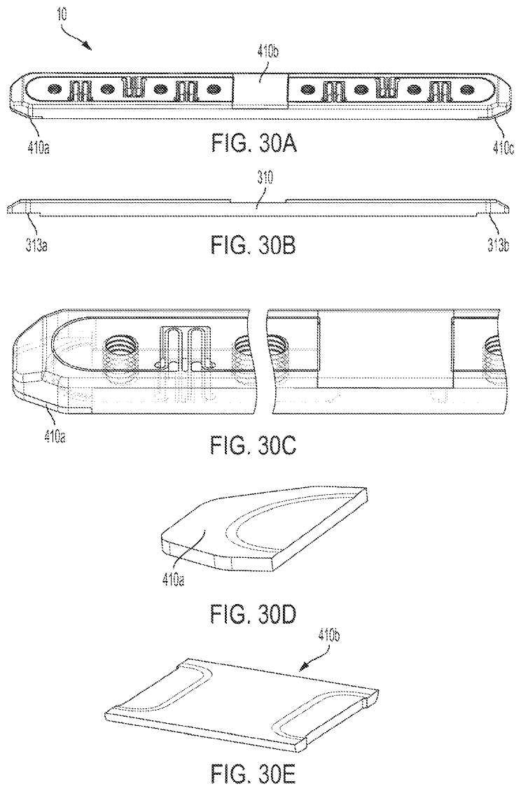

FIGS. 30A-30E show bone plates incorporating multiple rigid support plates connected to the static outer frame, according to embodiments of the present disclosure. Specifically, FIGS. 30A-30E show how multiple, smaller rigid support plates can be connected to the outer frame to prevent rotation at the ends of the suspended bodies. Since the most proximal and distal ends of each suspended body will exhibit the greatest out of plane translation, it is these portions of the suspended bodies that should be prevented from rotating out of plane. In the embodiments shown in FIGS. 30A-30E, small portions of the outer frame are removed 313a, 313c and support plates 410a, 410c are attached to those regions which extend beyond the original outer frame directly above or below the suspended bodies, preventing their rotation above or below the outer frame. In addition, support plate 410b can cover a central region of the compliant bone plate (e.g., near the fracture), and only partially overlap the suspended fixation bodies. Advantages of including multiple smaller support plates as opposed to a single support plate spanning the whole plate length, include the ability to maintain full cross-sectional thickness of the outer frame, suspended bodies, and flexures through much of the plate's length; only at the ends of the plate and near the center of the plate must the suspended bodies be thinner to allow insertion of the support plates without increasing the total plate thickness. Such support plates could be attached to the outer frame with welding such as tungsten arc welding, spot welding, laser welding, assembly with pins or screws or other generic assembly mechanisms, or any other method known in the art.

FIGS. 31A-31F show embodiments and results for support plate feature, according to an embodiment of the disclosure. FIGS. 31A and 31B show how one or more support plates can be incorporated directly into the design of the plate, maintaining a single piece part count for the plate, through methods such as additive manufacturing. FIGS. 31C and 31D show top and bottom views of a compliant plate with a support plate that is attached via pins or screws through the outer frame. FIGS. 31E and 31F show preliminary results for a flexure-based compliant bone plate undergoing a bending simulation both with (FIG. 23H) and without (FIG. 23I) a support plate. The addition of the support plate 410 decreases the total displacement by nearly half.

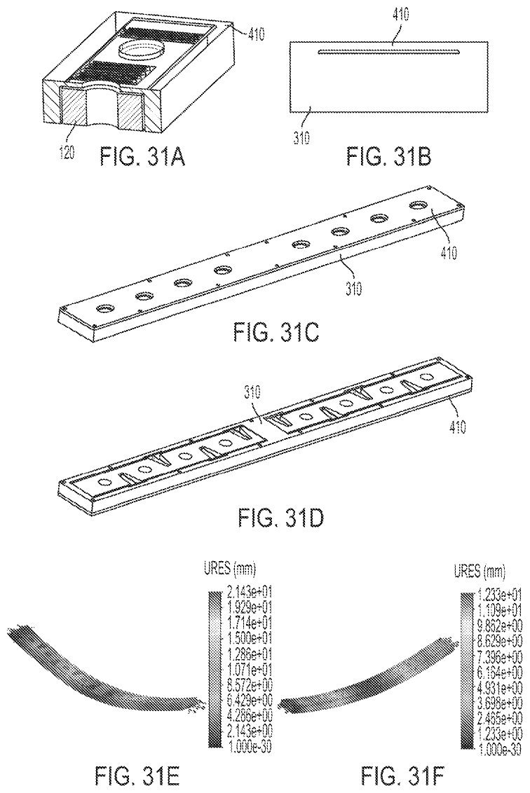

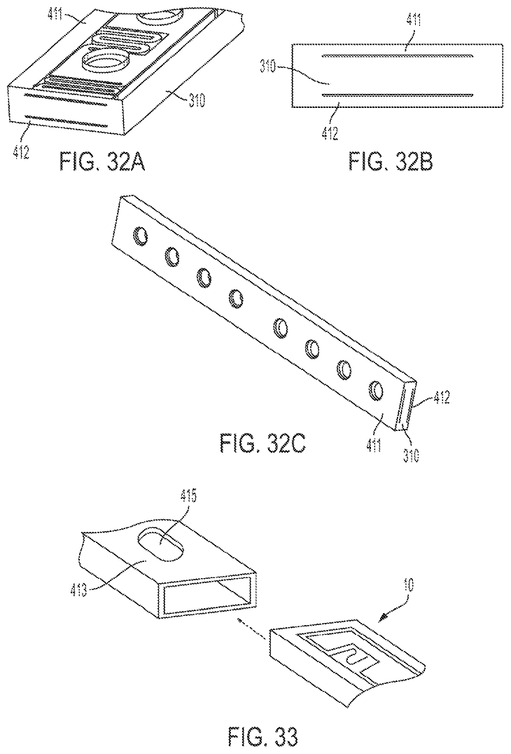

FIGS. 32A-32C illustrate an embodiment of a compliant bone plate having a dual cover, according to an embodiment of the disclosure. In some embodiments, a compliant bone plate can further include dual support plates 411, 412 for additional bending and torsion resistance. Thin support plates 411, 412 can be incorporated on both sides of the compliant bone plate (i.e., bone facing and non-bone facing). This can prevent out-of-plane bending of suspended fixation bodies relative to the outer frame 310 in all directions and can provide additional rigidity to the outer frame itself when the support plates 411, 412 are connected.

In either single support plate or dual support plate embodiments, the addition of the one or more support plates can either increase the thickness (prominence) of the compliant bone plate or maintain this thickness but reduce the thickness used in the compliant members within the support plates. This is an important consideration since axial stiffness of the compliant bone plate can be linearly correlated to the width of the flexures.

FIG. 33 illustrates an embodiment of a compliant bone plate being inserted into a rigid outer shell 413, according to an embodiment of the disclosure. In some embodiments, a compliant bone plate 10 may be inserted into a rigid outer shell 413 before being affixed to bone fragments. The rigid outer shell 413 can provide for additional bending and torsion resistance. This outer shell 413 can prevent bending out-of-plane of the suspended fixation bodies.

In some embodiments, the outer shell 413 and/or support plates 410, 411, 412 can comprise oblong holes 415 to allow for the screws (which attach only to the suspended fixation bodies) to translate axially relative to the outer frame 310 and the outer shell 413 or support plate 410, as applicable.

FIGS. 50A-50C show various ways one or more support plates can be connected to a compliant bone plate, according to embodiments of the present disclosure. As one of ordinary skill in the art will appreciate, one or more support plates can be on the top or bottom of a compliant bone plate, both the top and bottom, and at multiple locations on the frame, examples of which are illustrated in FIGS. 50A-50C. It could be advantageous to place one support plate on the top and another on the bottom as illustrated in FIG. 50C considering the suspended bodies can rotate out-of-plane from the outer frame in either direction. So only one cover plate may prevent displacement on one side but not the other.

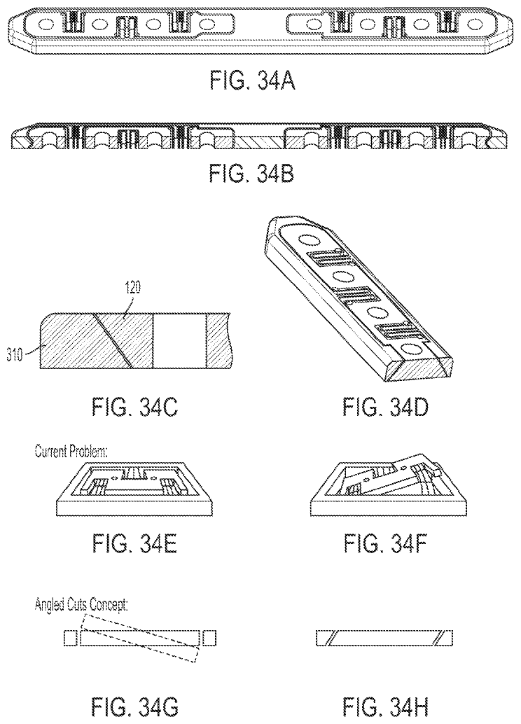

FIGS. 34A-34H illustrate the need for a compliant bone plate with angled cuts, according to an embodiment of the present disclosure. Angled cuts, such as those shown in FIGS. 34A-34H can prevent the out of plane motion of the suspended bodies relative to the outer frame. This feature (i.e., the angled cuts) addresses one potential challenge of utilizing a flexure-based bone plate, which is that large bending and torsional loads applied to the screw holes of the plate can cause the suspended bodies to rotate out of plane from the outer frame (see FIG. 34G). This phenomenon reduces the torsional and bending stiffness of a bone construct treated with the proposed plates and it results in undesired amounts of strain on the flexures which bend and twist as the suspended body rotates.

The angled cuts approach involves slots which extend from the bone-facing face of the plate to the non-bone-facing face of the plate and separate the suspended bodies from the outer frame, with some or all of the slots including angled portions such that the direction of the slot is non-perpendicular to the bone- or non-bone-facing face of the plate. By angling the cuts, any out of plane rotation of the suspended bodies results in contact between the suspended body and the outer frame, engaging the outer frame and stiffening the plate. Angled portions of the slots can be present at any location along the slot's path. Such angled cuts can be manufactured using wire EDM with an angled wire, angled laser cutting, water jetting, or milling, additive manufacturing, or other methods. Angled cuts can be made at angles such that the rotation of the suspended bodies is prevented in both directions (varus and valgus bending) or prevented only in the dominant mode of rotational motion (varus bending). Advantages of the angled cuts designs include the fact that the design remains a single-piece design, eliminating any assembly. Angled cut designs can be combined with any of the flexure types or other bending and torsion rigidity reinforcement features described herein.

FIGS. 35A-35F illustrate various views of a compliant bone plate comprising longitudinal slots, according to an embodiment of the disclosure. In some embodiments, a compliant bone plate may include longitudinal slots 520a, 520b that interface with corresponding protrusions 530a, 530b of the suspended fixation bodies to guide axial motion and prevent out-of-plane bending of suspended fixation bodies. Longitudinal features such as slots or grooves 520a, 520b can be incorporated to restrict motion to be purely axial between the suspended fixation bodies and the outer frame. As a bending load is applied physiologically to the bone and transferred through the screws to the suspended fixation bodies, the suspended fixation bodies can be constrained to stay in-plane with the outer frame due to the geometry of the longitudinal slots or grooves 520a, 520b.

FIGS. 35A and 35B show rectangular slots and grooves while FIGS. 35E and 35F show V-shaped slots and grooves. The advantage of V-shaped slots and grooves is that they can be manufactured with additive manufacturing without the use of support material and still be constructed of a single-piece. With an angled groove overhang of less than 45 degrees, these features are desirable with regards to using additive manufacturing without support material or post-printing assembly.

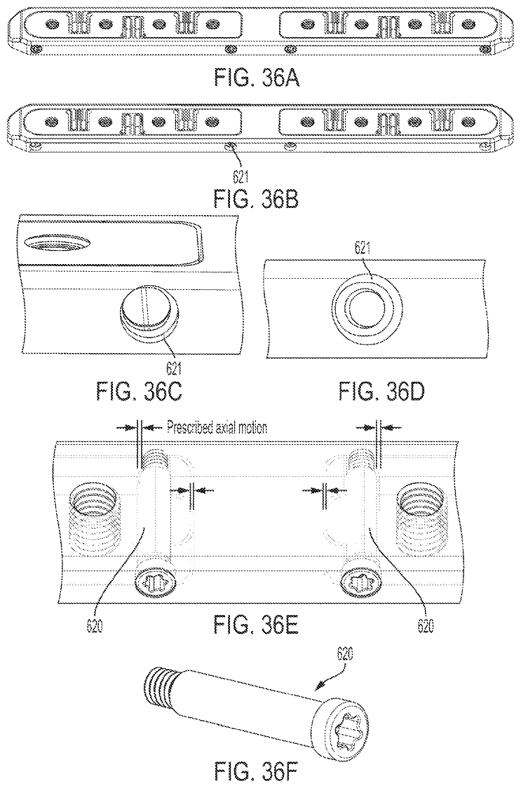

FIGS. 36A-36F illustrate various views of a compliant bone plate comprising a transverse pin 620, according to an embodiment of the disclosure. In some embodiments, a compliant bone plate may include one or more transverse pins 620 to prevent torsion or bending out-of-plane. In some embodiments, a transverse pin 620 can be connected rigidly to the outer frame and be located within an oblong thru hole 621 through a suspended fixation body. Because the thru hole 621 in the suspended fixation body is oblong, the suspended fixation body can translate purely axially without interfering with the rod 620, which can be cylindrical. However, as a bending or torsional load is applied to the compliant bone plate, the lateral guiding rod 620 can prevent the out-of-plane rotation or translation of the suspended fixation body relative to the outer frame.

In FIGS. 36A-36F, the transverse pins are shown as having threads distally which mate with a threaded thru hole on the far side of the outer frame. The near side of the outer frame contains a countersink feature for the pin head to sit flush into. Once the transverse pin is tightened down, it remains concentric to the side of the oblong slot that is nearest the center of the plate. The length of the oblong slot is approximately equal to the width of the prescribed axial motion gap between the suspended body and outer frame.

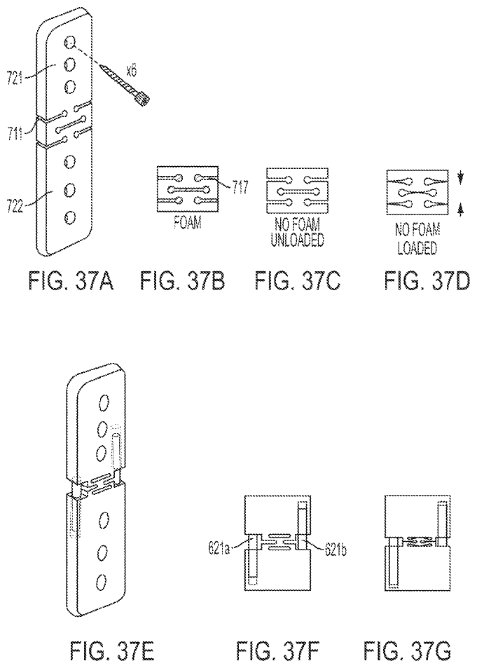

FIGS. 37A-37G illustrate embodiments of a compliant bone plate without an outer frame, according to an embodiment of the disclosure. In some embodiments, a compliant bone plate may not have an outer frame. Rather, in some embodiments, a compliant bone plate comprises two fixation bodies 721, 722 connected to each other via a flexure 711, as illustrated in FIGS. 37A and 37E. In these embodiments, flexures 711 could assume topologies of S shape, curved flexures, lattices, polygon mesostructures, LET joints, or others.

As illustrated in FIGS. 37B-37D, in some embodiments, bioabsorbable foam 717 can be added to the negative space regions of the flexures to provide additional bending and torsion resistance initially (see, for example, FIG. 37B) and to provide larger axial stiffness after implantation which decreases as a function of time (see, for example, FIG. 37D). Further, as shown in FIGS. 37E-37G, in some embodiments, a compliant bone plate can include one or more longitudinal guiding rods 621a, 621b to guide axial motion.

The embodiments described above can be attached to a bone via traditional conventional or locking screws. However, the subject matter disclosed herein is not so limited. Rather, as one of ordinary skill in the art will appreciate, the compliant bone plate can be attached to bone fragments any way that is known in the art.

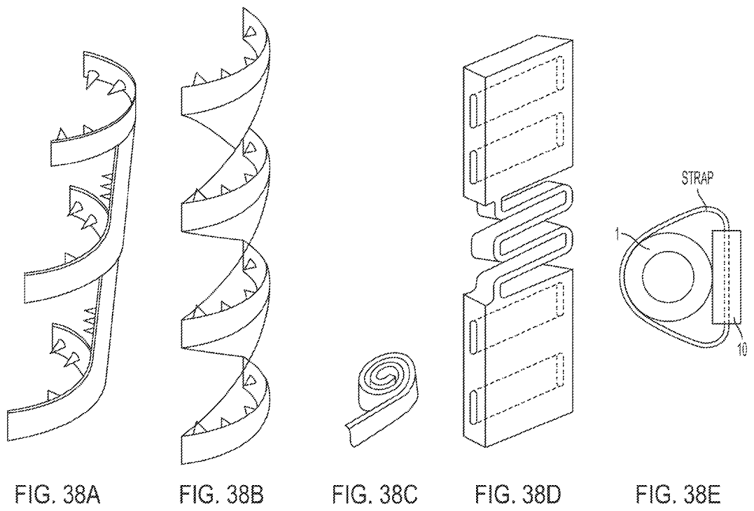

FIGS. 38A-38E illustrate compliant plates which affix to bone with methods other than screws, according to embodiments of the disclosure.

The embodiments described herein can attach to a bone with methods other than traditional conventional or locking screws. For example, straps with or without spikes can be slid through slots in the plate and secured back to the bone plate after wrapping around the bone, as illustrated in FIGS. 38C-38E. Another example is a plate that wraps around the bone with build-in spikes for purchasing onto the bone, embodiments of which are illustrated in FIGS. 38A and 38B.

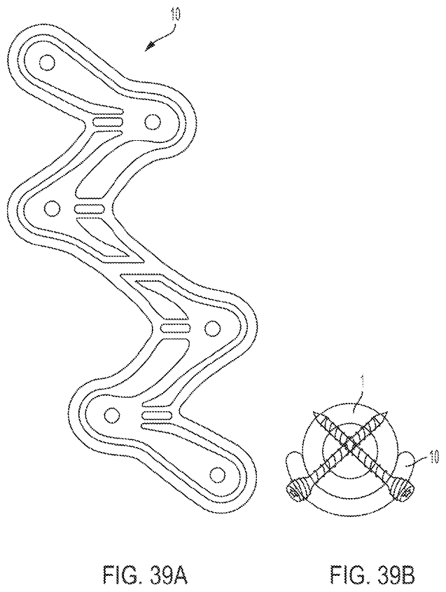

FIGS. 39A and 39B illustrate a compliant bone plate configured to wrap around bone, according to an embodiment of the disclosure. In some embodiments, a compliant bone plate 10 can wrap around the bone 1 to increase bone purchase and improve bending and torsion resistance. As shown in FIGS. 39A and 39B, a compliant bone plate 10 can assume a non-rectangular shape with regions of negative space to preserve blood supply to the healing bone while still incorporating screws to be attached to the bone 1.

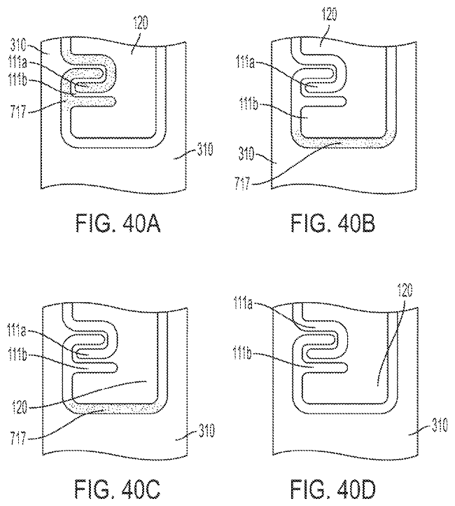

FIGS. 40A-40D illustrate a compliant bone plate with (FIGS. 40A-40C) and without (FIG. 40D) an insert in the negative regions around the suspended fixation body, according to embodiments of the disclosure. In some embodiments, a compliant bone plate may further comprise one or more bioabsorbable inserts 717 for controlled force-deflection response of a compliant bone plate as a function of time. In some embodiments, the bioabsorbable insert 717 can comprise a bioabsorbable material. In some embodiments, the bioabsorbable material can comprise a bioresorbable foam. In some embodiments, a bioabsorbable material 717 can be inserted into to the negative space regions adjacent to the suspended bodies such as in the prescribed motion gap or around the flexures 111 to provide additional bending and torsion resistance initially and to provide larger axial stiffness after implantation, which can decrease as a function of time as the bioresorbable material degrades.

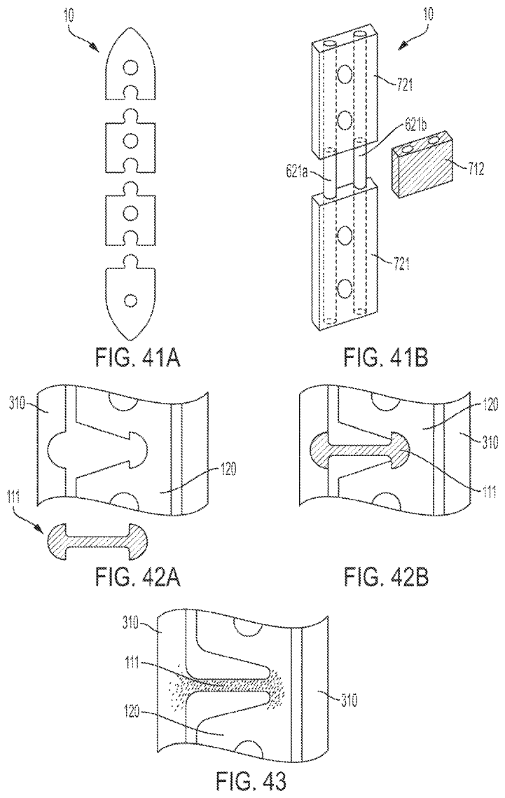

FIGS. 41A and 41B illustrate embodiments of modular (“puzzle piece”) flexible compliant bone plates, according to embodiments of the present disclosure. In some embodiments, a compliant plate can be modular, according to embodiments of the disclosure. In some embodiments, as illustrated in FIG. 41A, each piece of the compliant bone plate can connect to another piece in a similar way to how puzzle pieces fit together, as illustrated in FIG. 41A. In some embodiments, a compliant bone plate can be constructed from the modular assembly of plate components with different stiffnesses. For example, a rigid proximal plate 721 can be connected to a rigid distal plate 722 with guiding rods 621a, 621b connecting the two, and a space between the two plates 721, 722. A segment with a lower stiffness 712 can be added between the two plates 721, 722, which can mate to the rods 621a, 621b, providing the dynamic compression. This lower stiffness segment 712 can be monolithic, with the low stiffness resulting from a more flexible material or a compliant flexure design using the same rigid material as the rest of the plate.

FIGS. 42A and 42B illustrate an embodiment of a compliant plate that incorporates multiple modular components, according to an embodiment of the disclosure. In some embodiments, a compliant bone plate can incorporate multiple modular components that can comprise, for example, different surface finishes, porosities, and material compositions. As illustrated in FIGS. 42A and 42B, flexures 111 can be added to compliant bone plates in a modular fashion. For example, a compliant bone plate may be desired to be made from stainless steel due to its low osteoconductive properties, low cost, or ease of manufacturing. However, if a very large range of motion is required, a certain composition or surface finish of stainless steel may not be ideal for use in flexures required to deflect large amounts. For example, an alternative material composition, alloy, porosity, or machining process with improved surface finish can be used to make the flexures 111, which are assembled into the plate via welding, fasteners, etc., as illustrated in FIGS. 42A and 42B. For example, in some embodiments, a titanium alloy may be used for the flexure 111. This approach could reduce cost, allowing for high-precision processes, treatments, materials, etc. to be used selectively in areas undergoing large deflections and stresses rather than globally.

FIG. 43 illustrates an embodiment of a compliant plate with multiple materials, according to an embodiment of the disclosure. In some embodiments, a compliant bone plate can incorporate multiple materials via additive manufacturing voxel-based or other approaches. As shown in FIG. 43, multiple materials can be incorporated into various portions of the compliant plate (such as the flexures) via additive manufacturing methods. These can include voxel-based approaches (see FIG. 43) or material jetting, etc. depending on the types of materials desired for use in the compliant bone plate.

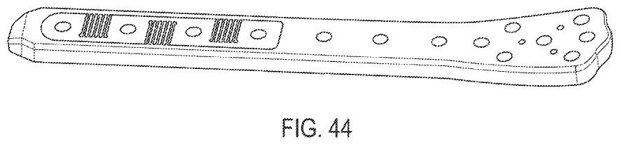

FIG. 44 shows a compliant metaphyseal bone plate, according to an embodiment of the present disclosure. FIG. 44 shows a metaphyseal embodiment of the present disclosure, in which the plate can possess a non-uniform cross section and a non-straight longitudinal axis. For example, proximal humerus plates, distal femur plates, and proximal tibia plates all typically possess similar outer geometry to that shown in FIG. 44. The flexible elements can be present on one or both sides of the plate. In FIG. 44, flexures are only present on the mid-shaft side of the plate where the bone is cylindrical. On the other side of the plate, a higher density of screw holes is present which is desirable clinically for fixing to the metaphysis of a long bone.

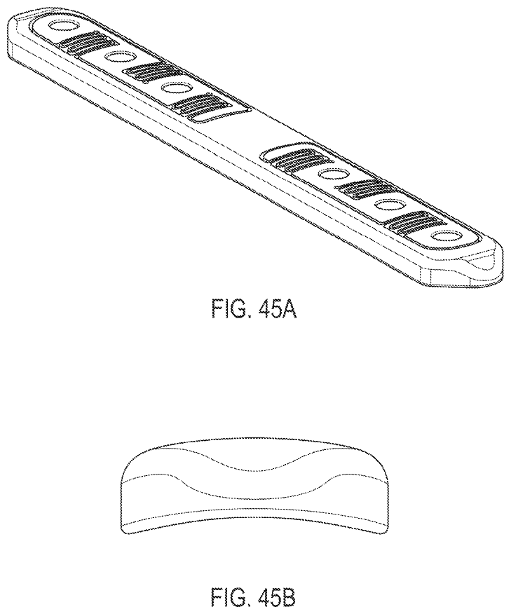

FIGS. 45A and 45B show various views of a crescent cross-section compliant plate, according to an embodiment of the present disclosure. This cross-section can allow better contouring of the plate to the bone and reduce the prominence of the plate.

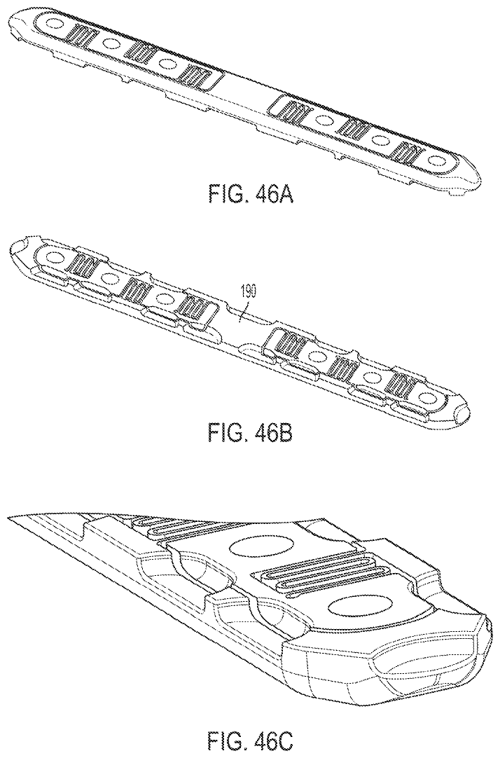

FIGS. 46A-46C illustrate various views of an embodiment of a compliant bone plate having limited contact features, according to embodiments of the disclosure. FIG. 46A illustrates the face of the compliant bone plate that does not contact the bone, and FIG. 46A illustrates the face of the compliant bone plate the does contact the bone. In some embodiments, the compliant bone plate has a limited contact feature 190. The limited contact feature 190 can be on the face of the compliant bone plate that contacts the bone (shown in FIG. 45B). As illustrated in FIGS. 46A-46C, in some embodiments, the limited contact feature 190 can include dimples or a narrow feature that contacts the bone instead of a solid face contacting the entire bone. In some embodiments, the limited contact feature 190 can involve scalloped features or flute cuts at locations which remove material from the suspended bodies and/or outer frame while preserving the screw hole and flexure geometry. This can be advantageous because it can reduce the surface area contacting the bone, increasing the blood supply to the healing bone.

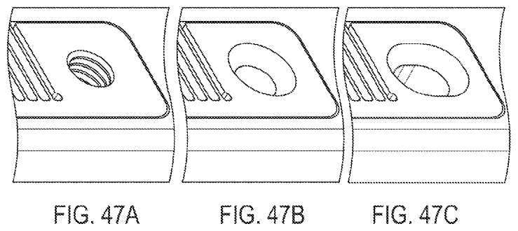

FIGS. 47A-47C show screw holes consisting of either locked (threaded) holes, conventional compression (chamfered countersunk) holes, or conventional oblong compression (chamfered countersunk) holes, respectively, and according to embodiments of the present disclosure. FIGS. 47A-47C illustrate how the present disclosure can consist of any combination of locked holes with threads for engaging locking screws (FIG. 47A), conventional compression holes for compression screws, including a countersink feature (FIG. 47B), or conventional oblong compression holes with countersink feature (FIG. 47C). These different hole designs can allow the plate to be compatible with locking or conventional screws for internal fixation as a locking plate or compression plating, if desired by the surgeon.

FIGS. 48A-48E illustrate examples of manufacturing methods for manufacturing a compliant bone plate, according to embodiments of the disclosure. In some embodiments, a compliant bone plate is manufactured by laser cutting (FIG. 48A), wire electrical discharge machining (EDM, FIG. 48B), and water jetting (FIGS. 48C and 48D). The production feasibility of the compliant bone plates disclosed herein have been investigated using multiple subtractive manufacturing methods. Many of the compliant bone plates incorporate planar designs that can be produced using top-down manufacturing methods. Many designs also involve slender flexible regions (e.g., flexures, lattices, etc.) that can be required to be made very thin (0.15-2 mm) depending on the flexure topology, material, boundary conditions, and required amount of deflection. In some embodiments, laser cutting and water jetting offer promising options for subtracting material around thin flexures. In other embodiments, wire EDM can be used for this purpose. Using wire EDM to manufacture a flexure-based compliant bone plate can offer many advantages due to its absence of turbulence, high precision, high accuracy, and the availability of very small wires to create very thin cuts.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure that are within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 components refers to groups having 1, 2, or 3 components. Similarly, a group having 1-5 components refers to groups having 1, 2, 3, 4, or 5 components, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A compliant bone plate configured to be attached to at least two bone fragments, the compliant bone plate comprising:
a frame forming therethrough one or more frame apertures, wherein an inner wall of the frame formed by a first frame aperture of the one or more frame apertures comprises a first wall, a second wall opposite the first wall, a third wall, and a fourth wall opposite the third wall;
a first suspended fixation body (“SFB”) within a first frame aperture of the one or more of the one or more frame apertures, wherein an outer face of the first SFB comprises a first face, a second face opposite the first face, a third face, and a fourth face opposite the third face;
a first flexure connecting the third wall of the frame to the third face of the first SFB; and
a second flexure connecting the fourth wall of the frame to the fourth face of the first SFB.

2. The compliant bone plate of claim 1, wherein a respective axial movement of the first SFB within the first frame aperture is limited by a distance between the outer face of the first SFB and the inner wall of the first frame aperture.

3. The compliant bone plate of claim 1,
wherein the first SFB is configured to attach to a first of the at least two bone fragments, and
wherein the frame is configured to attach to a second of the at least two bone fragments.

4. The compliant bone plate of claim 1, further comprising:
a second SFB within a second of the one or more frame apertures configured to attach to a second of the at least two bone fragments.

5. The compliant bone plate of claim 1, wherein the first SFB forms therethrough a plurality of attachment apertures.

6. The compliant bone plate of claim 1, wherein the first SFB forms therethrough a first set of one or more attachment apertures and is configured to attach to a first of the at least two bone fragments, the compliant bone plate further comprising:
a second SFB within one of the one or more frame apertures, wherein the second SFB forms therethrough a second set of one or more attachment apertures and is configured to attach to a second of the at least two bone fragments,
wherein the compliant bone plate is configured to be attached to the at least two bone fragments only at the one or more attachments apertures in the first and second SFBs.

7. The compliant bone plate of claim 1,
wherein the compliant bone plate is configured to promote secondary healing of the at least two bone fragments by permitting axial movement of the first SFB within the first frame aperture.

8. The compliant bone plate of claim 1,
wherein the first and second flexures can be selectively placed from a first configuration in which the first and second flexures are at rest to a second configuration in which the first and second flexures are pre-strained, and
wherein when the compliant bone plate is installed with the first and second flexures in the second configuration, the compliant bone plate promotes primary healing by compressing the at least two bone fragments together as the first and second flexures return to the first configuration.

9. The compliant bone plate of claim 1,
wherein the first SFB forms therethrough a cutout, and
wherein at least one of the first and second flexures is connected to the first SFB in the cutout.

10. The compliant bone plate of claim 1, further comprising:
a lateral guiding rod extending through at least part of the frame and at least part of the first SFB.

11. The compliant bone plate of claim 1, wherein each of the frame and the first SFB comprise a respective thicker first area and a respective thinner second area, the compliant bone plate further comprising:
a support plate proximate the respective thinner second areas of the frame and the first SFB to prevent out-of-plane rotation of the first SFB relative to the frame.

12. A compliant bone plate configured to be attached to at least two bone fragments, the compliant bone plate comprising:
a frame forming therethrough one or more frame apertures;
one or more suspended fixation bodies (“SFBs”) within one or more of the one or more frame apertures, wherein each of the one or more of the SFBs form therethrough one or more attachment apertures, and wherein each of the one or more SFBs configured to attach to a respective bone fragment such that no more than one of the one or more SFBs is attached to a respective bone fragment of the at least two bone fragments; and
one or more flexures, each flexure connecting a respective one of the one or more SFBs to the frame,
wherein the compliant bone plate is configured to be attached to the at least two bone fragments only at the one or more attachments apertures in the one or more SFBs.

13. The compliant bone plate of claim 12,
wherein each of the one or more SFBs is within a respective frame aperture, and
wherein a respective axial movement of a respective SFB within a respective frame aperture of the one or more frame apertures is limited by a distance between a respective outer face of the respective SFB and a respective wall of the respective frame aperture.

14. The compliant bone plate of claim 12, wherein at least one of the one or more SFBs forms therethrough a plurality of attachment apertures.

15. The compliant bone plate of claim 12,
wherein an inner wall of the frame formed by a first of the one of the one or more frame apertures and comprises an upper wall, a lower wall, a right side wall, and a left side wall,
wherein the one or more SFBs comprises a first SFB within a first frame aperture of the one or more frame apertures, wherein an outer face of the first SFB comprises a top face, a bottom face, a right face, and a left face, and
wherein the one or more flexures comprises:
a first flexure connecting the right side wall of the frame to the right face of the first SFB, and
a second flexure connecting the left side wall of the frame to the left face of the first SFB.

16. The compliant bone plate of claim 12,
wherein a first SFB of the one or more SFBs forms therethrough a cutout, and
wherein at least one of the one or more flexures is connected to the first SFB in the cutout.

17. A compliant bone plate configured to be attached to at least two bone fragments, the compliant bone plate comprising:
a frame forming therethrough one or more frame apertures;
one or more suspended fixation bodies (“SFBs”) within one or more of the one or more frame apertures; and
one or more flexures, each of the one or more flexures connecting a respective one of the one or more SFBs to the frame, wherein the one or more flexures comprise at least one of a switchback turn, an LET joint, a curved portion, and a plurality of linear flexures in parallel.

18. The compliant bone plate of claim 17,
wherein each of the one or more SFBs is within a respective frame aperture, and
wherein a respective axial movement of a respective SFB of the one or more SFBs within a respective frame aperture of the one or more frame apertures is limited by a distance between a respective outer face of the respective SFB and a respective wall of the respective frame aperture.

19. The compliant bone plate of claim 17,
wherein each of the one or more SFBs configured to attach to a respective bone fragment such that no more than one of the one or more SFBs is attached to a respective bone fragment of the at least two bone fragments, and
wherein each of the one or more SFBs forms therethrough a plurality of attachment apertures and the compliant bone plate is configured to attach to the at least two bone fragments only at the plurality of attachments apertures in the one or more SFBs.

20. The compliant bone plate of claim 17,
wherein an inner wall of the frame formed by a first of the one of the one or more frame apertures and comprises an upper wall, a lower wall, a right side wall, and a left side wall,
wherein a first SFB of the one or more SFBs within a first frame aperture of the one or more frame apertures, wherein an outer face of the first SFB comprises a top face, a bottom face, a right face, and a left face, and
wherein the one or more flexures comprises:
a first flexure connecting the right side wall of the frame to the right face of the first SFB, and
a second flexure connecting the left side wall of the frame to the left face of the first SFB.
